(19) United States
(12) Patent Application Publication
(10) Pub. No.: US 2025/0352360 A1
(43) Pub. Date: Nov. 20, 2025

(54) INFLATABLE PROSTHESIS, DELIVERY TOOLS THEREFOR, IMPLANTATION METHOD THEREFOR, AND MANUFACTURING METHOD THEREFOR

(71) Applicant: Stryker Corporation, Kalamazoo, MI (US)

(72) Inventors: Edson Falcao Lopes, San Jose, CA (US); Kyle Craig Pilgeram, San Jose, CA (US); Omar Elmalek, Caesarea (IL); Yoav Meiraz, Avihail (IL); James Flom, San Carlos, CA (US)

(73) Assignee: Stryker Corporation, Kalamazoo, MI (US)

(21) Appl. No.: 19/280,938

(22) Filed: Jul. 25, 2025

Related U.S. Application Data
(63) Continuation-in-part of application No. 18/663,772, filed on May 14, 2024.
(60) Provisional application No. 63/466,508, filed on May 15, 2023.

(51) Int. Cl. A61F 2/46; A61F 2/30

(52) U.S. Cl. CPC A61F 2/4612; A61F 2/30734; A61F 2002/30019; A61F 2002/30736; A61F 2220/0008; A61F 2240/001; A61F 2310/00005

(57) ABSTRACT

A method of delivering a balloon implant into a patient includes delivering an augment to an implantation site within the patient, securing the augment to tissue when the augment is at the implantation site, and delivering a balloon implant including a balloon to the implantation site and inflating the balloon such that movement of the balloon implant is restrained based on a position of the augment

### CROSS REFERENCE TO RELATED APPLICATIONS

This application is a continuation-in-part of U.S. patent application Ser. No. 18/663,772, filed May 14, 2024, which claims the benefit of and priority to U.S. Provisional Patent Application No. 63/466,508, filed May 15, 2023, the disclosures of both of which are incorporated by reference herein in their entireties.

### BACKGROUND

Through repeated strenuous motion, soft tissues in the human body often suffer wear and tear injuries from repeatedly rubbing against one another and/or hard tissues, such as bone. Tears of rotator cuff tendons and articular capsule disintegration are examples of this type of injury. In addition, these tissues can be adversely affected by inflammation, infection, disease and/or genetic predispositions which lead to degeneration of these tissues. Severe or complete tears and deterioration of articulations (i.e., bodily joints), related tissues (such as tendons, ligaments, capsules, cartilage and bony parts), and other bodily elements (such as bursae, synovium and other membranes) may cause severe pain, hindered movement up to complete disability, joint parts dislocation, and other possible phenomena.

Some joint-related deteriorations can be amended by filling voids and spaces between tissues with volumetric fillers. For example, inflatable members, such as balloons, may be implanted at an injury site, e.g., a joint, such as the shoulder joint, the prostate, or the stomach. Once inserted at a site and subsequently inflated, such balloons can prevent friction between tissues and/or re-center or re-align anatomy such as the humeral head in order to alleviate pain and prevent inflammation.

Also, it is often desirable to deliver medicaments to the injury site. Inflatable balloons can be used for this purpose as well. However, delivery of a medicament poses numerous complications. For example, if the medicament is contained within the balloon, the medicament must be released from the balloon without compromising the structural integrity of the balloon. It is also desirable to deliver such medicament at a steady rate, which is difficult to accomplish.

Thus, there exists a need to provide an improved way to promote tissue thickening and/or growth and/or healing in combination with such inflatable balloons.

### BRIEF SUMMARY

Provided according to an aspect of a first embodiment is a prosthesis for a location within an internal space, comprising an inflatable implant and an augment disposed on the implant. The prosthesis comprises an insertion configuration, wherein the implant is deflated. The prosthesis comprises an implanted configuration, wherein the implant is inflated.

According to an aspect of the first embodiment, in the implanted configuration, the implant is configured to simulate a bursa when inflated. In an aspect, the implant comprises an inflation port configured to allow a fluid to enter an interior space of the implant and inflate the implant. In a further aspect, the fluid comprises at least one of saline, water, biomaterial, collagen, medicament, tissue-growth promoter, and/or a solution that contains organic or inorganic salt.

According to an aspect of the first embodiment, in the insertion configuration, the augment is wound around the implant. In an aspect, the augment is disposed within one or more walls of the implant. In an aspect, the augment is coupled to at least a portion of an exterior surface of the implant. In a further aspect, in the implanted configuration, the exterior surface of the implant includes at least one surface capable of overlaying a target area, wherein the augment is disposed on the at least one surface of the implant and capable of being positioned between the implant and the target area.

According to an aspect of the first embodiment, the augment is mechanically or chemically coupled to the implant. In a further aspect, the augment is mechanically coupled to the implant via at least one filament. In still a further aspect, the prosthesis further comprises a plurality of intersecting filaments configured to secure the prosthesis in the implanted configuration. In another aspect, the augment is chemically coupled to the implant via an adhesive. In a further aspect, the portion of the exterior surface comprises the adhesive disposed thereon. In still a further aspect, the adhesive comprises a fibrin glue, a cyanoacrylate, or combinations thereof.

According to an aspect of the first embodiment, the implant further comprises an inflation port configured to allow a fluid to enter an interior space of the implant and inflate the implant, the inflation port configured to close when the interior space is sufficiently filled with the fluid. In an aspect, the prosthesis is disposed on an implant delivery tool in the insertion configuration.

According to an aspect of the first embodiment, the prosthesis is disposed within the internal space in the implanted configuration. In an aspect, in each of the insertion configuration and the implanted configuration, the prosthesis is configured to fit within the internal space. In an aspect, the internal space is the joint space located between the glenoid fossa and the humeral head. In an aspect, the internal space is the subacromial space. In an aspect, the prosthesis is configured such that, when inserted into the location within the internal space, the augment is adjacent to a rotator cuff or an acromion. In a further aspect, the prosthesis is coupled to the rotator cuff or the acromion.

According to an aspect of the first embodiment, the implant comprises at least one of polycaprolactone (“PCL”), polycarbonate polyurethane (“PCU”), polyglycolide (“PGA”), polyhydroxybutyrate (“PHB”), plastarch material, polyetheretherketone (“PEEK”), zein, polylactic acid (“PLA”), polydioxanone (“PDO”), poly(L-lactic acid) (“PLLA”), poly(D-lactic acid) (“PDLA”), poly (DL-lactic acid) (“PDLLA”) and poly(lactic-co-glycolic acid) (“PLGA”). In an aspect, the augment comprises a fibrous material. In a further aspect, the fibrous material comprises collagen, cross-linked collagen, non-cross-linked collagen, reconstituted collagen, native collagen, human collagen, bovine collagen, xenograph collagen, a synthetic material, a polyester material, an absorbable material, an organic material, silk, or combinations thereof.

Further provided according to an aspect of the first embodiment is a method comprising providing a prosthesis in an insertion configuration and disposed on an implant delivery tool, the prosthesis comprising an implant and an augment disposed thereon. The method further comprises inserting the prosthesis in the insertion configuration into a location within an internal space. The method further comprises inflating the prosthesis in the insertion configuration to provide the prosthesis in an implanted configuration.

According to an aspect of the first embodiment, the implant delivery tool comprises a handle disposed at a proximal end of the implant delivery tool and configured to be grasped by a user and an implant rod extending from the handle toward a distal end of the implant delivery tool. Furthermore, in the step of inserting the prosthesis, the handle is positioned by the user such that the implant rod is positioned adjacent to the location within the internal space. In a further aspect, the method further comprises retracting the implant rod from a position adjacent to the location within the internal space, wherein the prosthesis remains in the location within the internal space.

According to an aspect of the first embodiment, the implant delivery tool comprises a fluid path between the proximal end and the distal end and a port at the proximal end and in fluidic communication with the fluid path. Furthermore, the step of inflating the prosthesis comprises injecting a fluid through the port and fluid path and into the implant.

The description above with respect to the first embodiment is equally applicable to the additional embodiments described below, except where particular differences are described. For instance, the prosthesis, the implant, the augment, and the method of the second and third embodiments may each independently be the same as or different than that of the first embodiment.

Provided according to an aspect of a second embodiment is a system for a prosthesis for a location within an internal space, the system comprising at least one inflatable implant and at least one augment disposable on the implant to form the prosthesis, the augment comprising a fibrous material. The prosthesis comprises an insertion configuration, wherein the implant is deflated. The prosthesis comprises an implanted configuration, wherein the implant is inflated.

According to an aspect of the second embodiment, the system further comprises filaments for mechanically coupling the augment to the implant, an implant delivery tool, and an augment delivery tool. The implant delivery tool comprises a handle disposed at a proximal end of the implant delivery tool and configured to be grasped by a user, an implant rod extending from the handle toward a distal end of the implant delivery tool, a fluid path between the proximal end and the distal end, and a port at the proximal end and in fluidic communication with the fluid path. The augment delivery tool comprises an augment sleeve configured to encapsulate the implant rod and an augment rod disposed parallel to the augment sleeve and configured to secure the prosthesis proximate to the distal end of the implant delivery tool.

Further provided according to an aspect of the second embodiment is a method comprising providing an implant in an insertion configuration and disposed within an implant delivery tool, providing an augment in an insertion configuration and disposed on an augment delivery tool, coupling the augment to the implant to form a prosthesis in an insertion configuration, inserting the prosthesis in the insertion configuration into a location within an internal space, and inflating the prosthesis in the insertion configuration to provide the prosthesis in an implanted configuration.

According to an aspect of the second embodiment, the implant delivery tool comprises a handle disposed at a proximal end of the implant delivery tool and configured to be grasped by a user and an implant rod extending from the handle toward a distal end of the implant delivery tool. Furthermore, in the step of inserting the prosthesis, the handle is positioned by the user such that the implant rod is positioned adjacent to the location within the internal space. In a further aspect, the implant delivery tool comprises a fluid path between the proximal end and the distal end and a port at the proximal end and in fluidic communication with the fluid path. Further, the step of inflating the prosthesis comprises injecting a fluid with the port through the fluid path and into the implant.

According to an aspect of the second embodiment, the step of coupling the augment to the implant and the step of inserting the prosthesis are performed simultaneously. In a further aspect, the augment delivery tool comprises an augment sleeve configured to encapsulate the rod of the implant delivery tool and an augment rod disposed parallel to the augment sleeve and configured to secure the prosthesis proximate to the distal end of the implant delivery tool. Further, the step of coupling the augment to the implant and the step of inserting the prosthesis each comprise guiding filaments through the augment sleeve, the implant, and the augment to mechanically couple the implant and the augment to one another.

Provided according to an aspect of a third embodiment is an apparatus comprising a prosthesis for a location within an internal space, an implant delivery tool, and an augment delivery tool comprising an augment rod. The prosthesis is in an insertion configuration and is disposed on the implant delivery tool. The prosthesis comprises an inflatable implant disposed on the implant delivery tool and an augment disposed on the augment rod of the augment delivery tool and configured to be disposed on the implant, the augment comprising a fibrous material. The prosthesis comprises an insertion configuration, wherein the implant is deflated. The prosthesis comprises an implanted configuration, wherein the implant is inflated.

According to an aspect of the third embodiment, the apparatus further comprises filaments for mechanically coupling the augment to the implant. In a further aspect, the filaments are removably coupled to an augment sleeve of the augment delivery tool. In an aspect, the implant delivery tool comprises a handle disposed at a proximal end of the implant delivery tool and configured to be grasped by a user, an implant rod extending from the handle toward a distal end of the implant delivery tool, a fluid path between the proximal end and the distal end, and a port at the proximal end and in fluidic communication with the fluid path. In a further aspect, the augment delivery tool comprises an augment sleeve configured to encapsulate the implant rod and the augment rod disposed parallel to the augment sleeve and configured to secure the prosthesis proximate to the distal end of the implant delivery tool.

In one aspect, the present disclosure relates to a dual implant prosthesis for implantation in a patient. In a first example of a first embodiment, a prosthesis for implantation in a patient includes a first inflatable implant and a second inflatable implant movably attached to the first inflatable implant. The first and second inflatable implants are implantable into a patient in an uninflated configuration and are inflatable into an inflated configuration at an implantation location.

In a second example of the first embodiment, the first inflatable implant of the first example may be a first size and the second inflatable implant may be a second size different from the first size. In a third example, the second inflatable implant of the first or second example may be movably attached to the first inflatable implant through a filament. In a fourth example, the second inflatable implant of the first or second example may be movably attached to the first inflatable implant through a supplemental material segment. In a fifth example, the first inflatable implant and the supplemental material segment of the fourth example may be made of the same material. In a sixth example, the prosthesis of any one of the first through fifth examples may be configured such that the first inflatable implant is in fluid communication with the second inflatable implant. In a seventh example, the prosthesis of any one of the first through sixth examples may be configured such that the first implant includes a first inflation port and the second implant includes a second inflation port. In an eighth example, the prosthesis of the seventh example may be configured such that the first inflation port is parallel to the second inflation port. In a ninth example, the prosthesis of the seventh example may be configured such that the first inflation port extends in a first direction from first implant and the second inflation port extends in a second direction from the second implant, the second direction being different from the first direction. In a tenth example, the prosthesis of the ninth example may be configured such that the second direction is opposite the first direction. In an eleventh example, the prosthesis of any one of the first through tenth examples may also include an augment such that the first inflatable implant and the second inflatable implant are disposed within a cavity of the augment.

In one aspect, the present disclosure relates to a prosthesis including an implant and a receiver assembly. In a first example of a first embodiment, a prosthesis includes a receiver body that defines an internal volume and an inflatable implant disposed within the internal volume of the receiver body. The receiver body includes a peripheral opening. The inflatable implant is configured to pass through the peripheral opening to enter the internal volume of the receiver body. Further, an increase in volume of the inflatable implant through inflation of the inflatable implant is accompanied by a commensurate increase in volume of the internal volume of the receiver body.

In a second example of the first embodiment, the receiver body of the first example may include a plurality of peripheral cutouts, each cutout of the plurality of peripheral cutouts being spaced apart from the others. In a third example, the prosthesis of the first or second example may include a receiver assembly that includes the receiver body and a second receiver body attached to the receiver body. The second receiver body may have a second inflatable implant disposed within an internal volume of the second receiver body. In a fourth example, the prosthesis of the third example may be configured such that the second receiver body is attached to the first receiver body through a supplemental material segment adhered to both the first and second receiver body. In a fifth example, the prosthesis of any one of the first through fourth examples may include an augment such that the receiver body is disposed within a cavity of the augment.

In one aspect, the present disclosure relates to a method of implanting a prosthesis in a patient where the prosthesis includes two implant components. In a first example of a first embodiment, a method includes: delivering a first implant into a first space proximate a joint in a patient; delivering a second implant into a second space proximate the joint of the patient, the second space being physically separate from the first space; inflating the first implant to increase an internal volume of the first implant; and inflating the second implant to increase an internal volume of the second implant. In this method, the first implant and the second implant are in an attached condition prior to inflating the first implant and the second implant.

In a second example of the first embodiment, the method of the first example may include delivering fluid into the respective first and second implants as part of inflating the first and second implants. In a third example, the method of the first or second examples may include attaching the first implant to the second implant prior to delivering either the first or second implant into the patient. In a fourth example, the method of the first or second examples may include delivering a receiver assembly into the joint of the patient, wherein the delivering of the first implant into the first space includes delivery of the first implant into an internal volume of a first body of the receiver assembly and the delivering of the second implant into the second space includes delivery of the second implant into an internal volume of a second body of the receiver assembly. In a fifth example, the method of the fourth example may include retrieval of the first implant and the second implant from outside of the patient for delivery into the patient after delivering the receiver assembly into the joint of the patient. In a sixth example, the method of any one of the first through third examples may include placing the first implant into a first body of a receiver assembly and the second implant into a second body of the receiver assembly prior to delivering either the first or second implant into the patient. In a seventh example, the method of any one of the first through sixth examples may be performed such that the first space is a subacromial space and the second space is a glenohumeral joint.

In one aspect, the present disclosure relates to a method for performing a surgical procedure using an augment and an implant. In a first example of a first embodiment, a method includes: delivering an augment into an implantation site within a patient using an implant delivery system; delivering an implant into the implantation site within the patient using the implant delivery system, the implant being in an insertion configuration enclosed by a portion of the implant delivery system; causing the augment to be released from the implant delivery system; causing the implant to be exposed from the portion of the implant delivery system so that the implant is inside a cavity of the augment; inflating the implant into an implantation configuration; and removing the implant delivery system from the implant.

In a second example of the first embodiment, the method of the first example may include delivering the augment into the implantation site using an augment delivery tool of the implant delivery system and delivering the implant into the implantation site using an implant delivery tool of the implant delivery system. In a third example, the method of the second example may include delivering the augment into the implantation site by having the cavity of the augment in receipt of the implant delivery tool and an augment sleeve sized to fit over the portion of the implant delivery system. In a fourth example, the method of the second example may include delivering the implant into the implantation site by having the implant enclosed in the portion of the implant delivery system, the portion being a slidable sheath. In a fifth example, the method of any one of the first through fourth examples may include delivering the augment into the implantation site while the implant is outside of the patient, prior to delivering the implant into the implantation site.

In a sixth example of the first embodiment, the method of the first example may include delivering the augment and the implant into the implantation site using an implant delivery tool of the implant delivery system. In a seventh example, the method of the sixth example may include, prior to delivering the augment and the implant, loading the implant and the augment onto the implant delivery tool such that the implant is enclosed within the portion of the implant delivery system, the portion being a sheath. In an eighth example, the method of the seventh example may include loading the augment onto the implant delivery tool including initially positioning the implant into the cavity of the augment such that both the implant and the augment are enclosed within the sheath. In a ninth example, the method of the seventh example may include loading the augment onto the implant delivery tool by positioning the augment over a portion of the implant delivery tool external to the sheath. In a tenth example, the method of any one of the first through ninth examples may include providing the implant including a first implant body attached to a second implant body prior to delivering the implant into the implantation site.

In a first example of a second embodiment of a method for performing a surgical procedure using an augment and an implant, the method includes: providing an implant in an insertion configuration and disposed within a movable sheath of an implant delivery tool; providing an augment on the implant delivery tool, the augment being separated from the implant by the sheath; inserting the implant delivery tool into a patient; manipulating the implant delivery tool to cause the augment to be released from the implant delivery tool and the implant to be exposed from within the sheath such that the implant is within a cavity of the augment; inflating the implant into an implantation configuration; and releasing the implant from the implant delivery tool.

In a second example of the second embodiment, the method of the first example may include manipulating the implant delivery tool through a first manipulation to cause the augment to be released and a second manipulation to cause the implant to be exposed from within the sheath. In a third example, the method of the second example may be performed such that the first manipulation and the second manipulation are different extents of the same relative movement of parts of the implant delivery tool. In a fourth example, the method of the first example may include providing the augment by positioning an augment delivery tool adjacent to the implant delivery tool and positioning the augment over an augment sleeve of the implant delivery tool and the augment delivery tool, the augment sleeve being disposed over the sheath and being movable with the sheath. In a fifth example, the method of any one of the first through fourth examples may include providing the implant in the insertion configuration and disposed within the sheath such that providing the implant includes providing a first implant body attached to a second implant body.

At least one aspect of the present disclosure relates to a method of delivering a balloon implant into a patient, the method including delivering an augment construct to an implantation site within the patient, the augment construct including an augment, securing the augment construct to tissue at the implantation site, and delivering a balloon implant including a balloon to the implantation site and inflating the balloon such that the augment construct positionally restrains movement of the balloon implant when the balloon is inflated. In some embodiments, the augment construct includes a plurality of filaments, each filament including ends attached to the augment, wherein the movement of the balloon implant is restrained by the plurality of filaments or the plurality of filaments in combination with the augment.

In some embodiments, the augment construct defines a cavity and one or more openings large enough to allow passage of the balloon implant in a collapsed configuration into the cavity, wherein delivering the balloon implant includes inserting the balloon implant into the cavity via a first opening of the one or more openings. In some embodiments, the balloon is inflated to a size that restricts the removal of the balloon implant from the cavity through any of the one or more openings. In some embodiments, the one or more openings include at least two openings. In some embodiments, the method further includes closing the first opening after inserting the balloon implant into the cavity.

In some embodiments, the augment construct defines a cavity and includes only a single opening suitable for delivering the balloon implant therethrough into the cavity, wherein delivering the balloon implant includes inserting the balloon implant into the cavity through the opening. In some embodiments, the method further includes closing the opening after inserting the balloon implant into the cavity. In some embodiments, closing the opening includes tensioning a filament. In some embodiments, tensioning a filament includes stitching the opening shut.

In some embodiments, the augment has a balloon-type structure with a plurality of peripheral cutouts, each cutout spaced apart from the other cutouts, and wherein the balloon-type structure defines a cavity accessible through each of the plurality of cutouts, wherein delivering the balloon implant includes inserting the balloon into the cavity through one of the plurality of cutouts.

At least one other aspect of the present disclosure relates to a prosthesis including an inflatable balloon implant and an augment construct securable to tissue at an implantation site and including an augment, the augment construct defining a receptacle configured to receive the balloon implant at the implantation site in a deflated condition and to restrain movement of the balloon implant after the balloon implant is inflated at the implantation site.

In some embodiments, the augment construct includes a plurality of filaments, each filament including ends attached to the augment, the augment and the plurality of filaments defining the receptacle therebetween. In some embodiments, the receptacle is a cavity accessible from a plurality of openings in the augment construct. In some embodiments, the receptacle is a cavity accessible from exactly two openings sized to receive the balloon implant in the deflated condition on opposite ends of the augment construct, the augment including closed edges extending between the two openings. In some embodiments, the receptacle is a cavity accessible from exactly one opening sized to receive the balloon implant in the deflated condition. In some embodiments, the augment includes a plurality of peripheral cutouts, each cutout spaced apart from the other cutouts, wherein the receptacle is a cavity accessible through each of the plurality of cutouts.

At least one other aspect of the present disclosure relates to a prosthesis including an inflatable balloon implant, an augment securable to tissue at an implantation site, and a structure attached to the augment, wherein the structure or the structure in combination with the augment defines a receptacle configured to receive the balloon implant at the implantation site in a deflated condition, and wherein the structure is configured to restrain movement of the balloon implant after the balloon implant is inflated at the implantation site.

In some embodiments, the structure includes a plurality of filaments, each filament including ends attached to the augment, and wherein the balloon implant is restrained by the plurality of filaments or the plurality of filaments in combination with the augment after the balloon implant has been inflated. In some embodiments, the structure includes a receiver assembly with a plurality of peripheral cutouts, each cutout spaced apart from the other cutouts, and wherein the receiver assembly defines a cavity accessible through at least one of the plurality of cutouts, wherein delivering the balloon implant includes inserting the balloon into the cavity through one of the plurality of cutouts.

### BRIEF DESCRIPTION OF THE DRAWINGS

FIG. 1 shows two prostheses according to one or more embodiments of the present disclosure.

FIG. 2 shows two prostheses according to one or more embodiments of the present disclosure.

FIG. 3 shows a plurality of apparatuses including implant delivery tools and prostheses according to the first embodiment of the present disclosure.

FIG. 4 shows an augment delivery tool according to the second and/or third embodiment of the present disclosure.

FIG. 5 is a model showing insertion of an augment delivery tool into an internal space according to the second and/or third embodiment of the present disclosure.

FIG. 6 is a model showing insertion of an implant delivery tool into an augment sleeve of an augment delivery tool according to the second and/or third embodiment of the present disclosure.

FIG. 7 is a model showing a system before retraction of an implant rod from an internal space according to the second and/or third embodiment of the present disclosure.

FIG. 8 is a model showing a system after retraction of an implant rod from an internal space according to the second and/or third embodiment of the present disclosure.

FIG. 9 is a model showing the prosthesis in the inflated configuration according to one or more embodiments of the present disclosure.

FIG. 10 shows an augment according to the second and/or third embodiments of the present disclosure.

FIGS. 11-18 show prostheses according to various embodiments of the present disclosure.

FIG. 19 shows a prosthesis implanted in a joint of a patient according to one embodiment of the present disclosure.

FIGS. 20 and 21 show a receiver assembly according to respective embodiments of the present disclosure.

FIGS. 22-24 show steps in a method of delivering an implant into a receiver assembly as part of a method of a joint repair according to one embodiment of the present disclosure.

FIGS. 25-29 show steps in method of delivering implant components into respective bodies of a receiver assembly as part of a method of a joint repair according to one embodiment of the present disclosure.

FIG. 30A shows an augment according to one embodiment of the present disclosure.

FIG. 30B shows an augment according to one embodiment of the present disclosure.

FIG. 31 is a model showing part of a system for delivering a prosthesis according to one embodiment of the present disclosure.

FIG. 32 is a cross-sectional view of the system of FIG. 31.

FIGS. 33-36 are steps in a method of implanting a prosthesis according to one embodiment of the present disclosure.

FIG. 37 is a flowchart illustrating a method of delivering a balloon implant according to one embodiment of the present disclosure.

FIGS. 38A-38E illustrate various aspects of the method of FIG. 37 according to one embodiment of the present disclosure.

FIG. 39A is a flowchart illustrating a method of delivering a balloon implant according to one embodiment of the present disclosure.

FIG. 39B is a flowchart illustrating a method of delivering a balloon implant according to one embodiment of the present disclosure.

FIG. 40 shows an augment according to one embodiment of the present disclosure.

FIG. 41 shows an augment according to one embodiment of the present disclosure.

FIG. 42 shows an augment according to one embodiment of the present disclosure.

FIGS. 43-46 illustrate various aspects of the method of FIG. 39A according to one embodiment of the present disclosure.

FIG. 47A shows a perspective view augment according to one embodiment of the present disclosure.

FIG. 47B shows a side cross-sectional view of the augment of FIG. 47A.

FIGS. 48-51 illustrate various aspects of the method of FIG. 39A according to one embodiment of the present disclosure.

FIG. 52 shows an augment according to one embodiment of the present disclosure.

### DETAILED DESCRIPTION

As described above, repeated strenuous motion often causes sensitive soft tissues associated with a human joint to suffer wear and tear injuries from repeatedly rubbing against one another and/or hard tissues. Injuries to soft tissues such as tendons can cause pain and impaired function of the area served by the tendon. Provided herein are various examples of a prosthesis for at least partially alleviating such pain and restoring function to impaired areas. In the illustrated embodiments, the prosthesis includes an implant, which is a biodegradable balloon capable of being inflated with a fluid, and an augment, which includes a collagen material or the like and is coupled to the implant. The implant may be included to, for example, reduce pain by imparting a cushion between damaged soft tissue and/or opposing bones in a joint, while the augment may be included to, for example, help promote tissue ingrowth and/or repair of the tissue against which it is positioned. While several examples disclosed herein refer to the treatment of shoulder joints (e.g., rotator cuff repair), the prostheses of the present disclosure are not limited to shoulder applications and may be used between any two or more tissues in a mammalian body where the placement would function to promote healing and/or restore anatomic function in a joint space.

In various embodiments, augments described herein may be made of or include at least one of collagen, cross-linked collagen, non-cross-linked collagen, reconstituted collagen, native collagen, human collagen, bovine collagen, xenograph collagen, a synthetic material (e.g., polymer), a polyester material, an absorbable or non-absorbable material, an organic material, silk, or combinations thereof. While the figures illustrate particular examples of augments of the present disclosure, it will be appreciated that this disclosure contemplates augments in any shape adapted to be received within a joint space. For example, in some examples, augment may be circular, square, rectangular, triangular, or any other simple or complex shape. The corners of an augment may be rounded or may be substantially squared. Further, in various embodiments, augments may include multiple individual pieces or sections of material.

As used herein, the term “augment construct” refers to a structure or assembly that includes at least an augment. In some embodiments, an augment construct may include only an augment. In other embodiments, an augment construct may include an augment as well as additional structural features. These additional structural features may include substrates for reinforcing the augment, sleeves, pockets, one or more filaments, or the like, configured to receive and restrain an implant, and/or features for restraining and/or securing the augment construct to tissue at an implantation site. The additional structural features may be made of or include different materials than the augment. For example, an augment construct may include a collagen augment coupled to a polymer substrate or to polymer filaments. Alternatively, the additional structural features may include the same material as the augment, such that the augment construct may include, for example, a collagen augment and a collagen sleeve or pocket; the collagen augment and the collagen sleeve or pocket may be separate components that can be coupled to one another, or may be a monolithic structure.

As used herein, the term “biodegradable” means able to be broken down and absorbed or eliminated by the human body.

As used herein, the term “bursa” means a naturally-occurring fluid-filled sac that acts to reduce impact and/or friction between moving structures in a joint. Since a bursa is typically found in high-friction or high-stress locations in a joint, such as in a shoulder joint, they are typically positioned near areas where joint injuries are prone to occur, which can also result in injuries to the bursa itself.

As used herein, the term “rotator cuff” means the group of muscles and their tendons that act to stabilize the shoulder and to permit rotation and abduction of the arm.

As used herein, nouns in the singular form encompass the plural form, and vice versa. For example, a “filament” may refer to one or more filaments, and “filaments” may refer to one or more filaments as well.

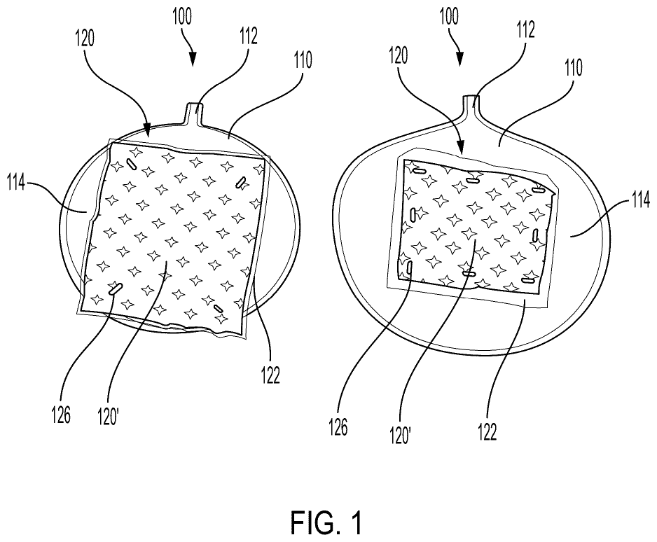

Referring to FIG. 1, a first embodiment of the present disclosure provides a prosthesis 100 for a location within an internal space. The prosthesis 100 includes an inflatable implant 110 and an augment 120′, which may be disposed on and/or coupled to the implant 110 and may be part of an augment construct 120 including additional components. The prosthesis 100 includes at least two configurations. In a first configuration, referred to herein as the insertion or deflated configuration, the implant 110 is deflated. Preferably, the augment 120′ may be associated with the implant 110 in the first configuration, or alternatively, at this stage may be separate from implant 110. For instance, in this first configuration, augment 120′ and implant 110 may be rolled up together in a way to consolidate the cross-sectional size and/or total volume of the prosthesis 100 which may allow for passage through a cannula or the like during surgery. Alternatively, augment 120′ may be wrapped around the deflated implant 110, or augment 120′ may be completely separate from deflated implant 110 such that each are rolled, or the like, separately. In a second configuration, referred to herein as the implanted or inflated configuration, which is shown in FIG. 1, the implant 110 is inflated. As shown in FIG. 1, the augment 120′ may be disposed on or coupled to at least a portion of an exterior surface of the implant 110. For example, augment 120′ may be coupled to implant 110 at this stage by adhering a surface of augment 120′ to a surface of implant 110. The prosthesis 100 may further include any number of additional configurations corresponding to partial inflations. For instance, the prosthesis 100 may exist in a partially inflated configuration while the implant 110 is being inflated during manufacture or implantation, whereby augment 120′ may be disposed on or coupled to implant 110 throughout, or may be, at some point, transitioned from being completely separate from implant 110 to being disposed on or coupled to implant 110.

In any configuration, the prosthesis 100 is configured to fit within an internal space. The prosthesis 100, including the implant 110 and the augment 120′, is initially disposed within the internal space while in the insertion or deflated configuration. Then the implant 110 is inflated to form the implanted configuration while remaining disposed in the internal space. Thus, the prosthesis 100 can be inserted into the internal space of a patient through a relatively small incision, and then the implant 110 and prosthesis 100 can be inflated to a larger volume appropriate for the internal space. Addition of the augment 120′ should not cause a dramatic increase to the incision size needed to insert the implant 110 and augment 120′ into the body.

The prosthesis 100 is configured such that, when inflated in the implanted configuration, the exterior surface of the implant 110 includes at least one surface capable of overlaying a target area. The augment 120′ is disposed on the exterior surface of the implant 110 and is capable of being positioned in between the implant 110 and the target area. The internal space and/or target area may be a joint space, a glenoid fossa, a humeral head, a subacromial space, a rotator cuff, an acromion, a damaged soft tissue, a damaged hard tissue, a space therebetween, and/or any other space within the internal anatomy of a patient. For example, the prosthesis 100 can be configured such that when inserted into the location within the internal space, the augment 120′ is positioned adjacent to the rotator cuff and opposite the acromion. Alternatively, the augment 120′ may be positioned adjacent to the acromion and opposite the rotator cuff. Collagen or another inert or bioactive material in the augment 120′ adjacent to the acromion may prevent bone to rotator cuff rubbing. Further, the augment 120′ or multiple augments 120′ may be positioned adjacent to both the acromion and the rotator cuff. The prosthesis 100 may be tethered to the rotator cuff or the acromion by a surgeon using one or more filaments after the prosthesis 100 is implanted into the internal space. The tethering may involve piercing the rotator cuff or acromion, hooking the filament onto the rotator cuff or acromion, or wrapping the filament around the rotator cuff or acromion, or any other suitable methods of tethering the filament to the rotator cuff, acromion, or another location within an internal space.

The filaments are preferably a biocompatible and biodegradable material. If the prosthesis 100 is tethered within the internal space, the tethering may also be performed with biocompatible and biodegradable attachments so that no materials adverse to human health are introduced to the body, and further such that all materials relating to the prosthesis 100, including such filaments and tethers, degrade within the patient. Though, in certain embodiments and applications, at least a portion of the prosthesis 100 and/or filaments/tethers may not be biodegradable if a more permanent device is preferred. Further, since the prosthesis 100 is inserted into the body in a deflated configuration, the attachments must be able to withstand inflation of the implant 110 without being damaged or lost.

As discussed above, the augment 120′ may be made from collagen, cross-linked collagen, non-cross-linked collagen, reconstituted collagen, native collagen, human collagen, bovine collagen, xenograph collagen, a synthetic material, a polyester material, an absorbable material, an organic material, silk, or combinations thereof. The implant 110 may be made from at least one of polycaprolactone (“PCL”), polycarbonate polyurethane (“PCU”), polyglycolide (“PGA”), polyhydroxybutyrate (“PHB”), plastarch material, polyetheretherketone (“PEEK”), zein, polylactic acid (“PLA”), polydioxanone (“PDO”), poly (L-lactic acid) (“PLLA”), poly(D-lactic acid) (“PDLA”), poly (DL-lactic acid) (“PDLLA”) and poly(lactic-co-glycolic acid) (“PLGA”).

According to an aspect of the present disclosure, the implant 110 is configured to simulate a bursa when inflated. In such aspects the implant 110 is shaped and sized to simulate the natural bursa found in the target implantation area within the internal space. As a specific example, the implant 110 may be configured to simulate the subacromial bursa of the shoulder.

The implant 110 includes an inflation port 112 configured to allow a fluid to enter an interior space 114 of the implant 110 and inflate the implant 110. The inflation port 112 is further configured to close when the interior space 114 is sufficiently filled with the fluid, i.e., in the implanted configuration. The implant 110 and inflation port 112 are further configured to release at least a portion of the fluid upon the application of pressure to the implant 110. The fluid may include at least one of saline, water, biomaterial, collagen, medicament, tissue-growth promoter, and/or a solution that contains organic or inorganic salt. For example, the fluid may be therapeutic fluid that may promote healing of the tissue at the implantation site, such as Hyaluronic acid, platelet-rich plasma, and/or bone marrow aspirate concentrate. The fluid in the implant 110 may be released from the balloon as the implant 110 biodegrades. The implant 110 possesses viscoelastic deforming characteristics such that the implant 110 gently resists pressure applied by parts of the human body during movement, and the implant 110 may flexibly expand and contract in response to changes in pressure applied externally to the implant 110. In some examples, implant 110 is the InSpace® balloon implant (Stryker Corporation, Greenwood Village, CO).

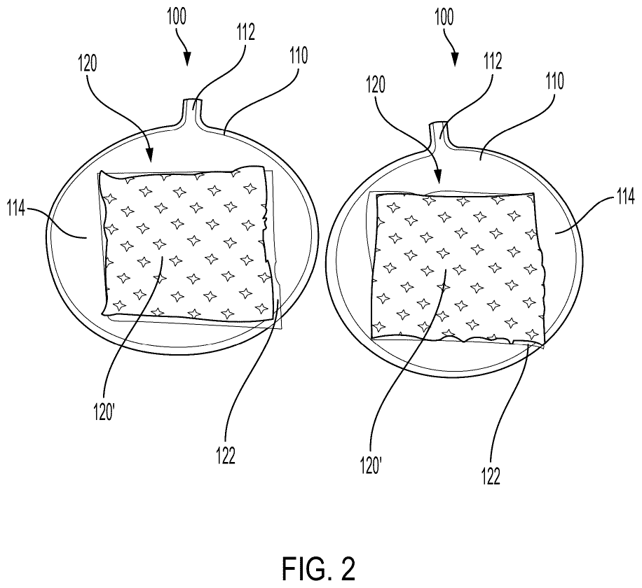

In the example shown in FIG. 1, the augment 120′ is disposed on the implant 110. More specifically, the augment 120′ may be mechanically or chemically coupled to the implant 110. Mechanical coupling may include the use of filaments 126, but is not limited thereto and includes any suitable methods known in the art. If filaments 126 are used, they may be biodegradable as described above, or alternatively may be permanent and biocompatible, so that in either case no harmful materials are introduced into the human body. For example, sutures commonly used in orthopedic applications, whether biodegradable or nonbiodegradable, may be used. As further described above, since the prosthesis 100 is inserted in a deflated configuration, the filaments 126 coupling the implant 110 and the augment 120′ must be able to withstand inflation of the implant 110 without being damaged or lost. Chemical coupling includes adhesion, but is not limited thereto and includes any suitable methods known in the art. In some aspects, the augment 120′ is coupled to at least a portion of an exterior surface of the implant 110. In a further specific aspect which is shown in FIG. 1, the augment 120′ is mechanically coupled to the exterior surface of the implant 110 via at least one filament 126. Examples of filament 126 may include sutures, biodegradable sutures, suture tape, or any other filament materials suitable for coupling the augment 120′ to the exterior surface of the implant 110. In some examples, the implant 110 and/or the augment 120′ may include tabs at their respective peripheries for coupling the implant 110 and the augment 120′ together (e.g., using filament, sutures, etc.). For example, the implant 110 may include multiple tabs spaced around the periphery of the balloon with openings extending therethrough. Filaments may be passed through the openings and secured to the tabs at respective first ends, and respective second ends of the filaments may be coupled to the augment 120′, for example, by passing the filaments through the material of the augment 120′ and securing the filament by tying a knot, forming a splice, or the like, with the filament around the edge of the augment 120′.According to an alternative aspect which is shown in FIG. 2, the augment 120′ or augment construct 120 is chemically coupled to the implant 110 via adhesion. The adhesive may include a fibrin glue, a cyanoacrylate, or combinations thereof. The adhesive may be applied in a form, such as a spray, wherein the augment 120′ or augment construct 120 is sprayed with the adhesive and attached to the surface of the implant 110, which leaves a thin layer of adhesive on the surface of the augment 120′ or augment construct 120. Such a spray may be an adhesive composition which is curable by light to form a thin adhesive resin binding the augment 120′ or augment construct 120 and implant 110 to one another. In a particular aspect, the adhesive composition is curable by ultraviolet (UV) or visible light. The adhesive composition may include additives such as photoinitiators for light curability. The curing reaction may be carried out over the course of several seconds, minutes, or hours. The rapid curing and curability by light allow the surgeon to choose precisely when the augment 120′ or augment construct 120 and implant 110 are adhered. The surgeon can use a light source to control the timing of adhesion, for instance by tuning a constant light source to the applicable wavelength of UV/visible light in order to initiate and/or accelerate curing. For example, a camera system having a tunable light source may be utilized during implantation, both to visualize the implantation procedure and to initiate and/or accelerate curing of the adhesive composition. Alternatively, a temporary light source with the applicable wavelength can be applied to the adhesive composition in order to initiate and/or accelerate curing. In some examples, the augment 120′ or augment construct 120 may be wetted with water and may adhere to the implant 110 without any further chemical or mechanical adhesives due to the respective material properties of the augment 120 and the implant 110. For example, the augment 120′ or augment construct 120 may be made of collagen, which may adhere to the implant 110 when wet.

In various aspects, a prosthesis may be implanted by securing an augment or augment construct to tissue at an implantation site in a patient, and then securing an implant to the augment or securing the implant to the tissue with the implant positioned on top of the augment (e.g., with the augment between the tissue and the implant). For example, referring to prosthesis 100 of FIGS. 1 and 2, augment 120′ or augment construct 120 may be secured to tissue at an implantation site using, for example, fixation members such as u-shaped bars, staples, pins, screws, etc. that may be pressed through the material of augment 120′ or augment construct 120, or passed through openings in augment 120′ or augment construct 120. In other examples, augment 120′ or augment construct 120 may be secured to tissue using filaments that may be secured directly to the tissue or to anchors installed into the tissue (e.g., for a double row repair of a rotator cuff). Implant 110 may then be inserted into the patient and secured to the augment 120′ or augment construct 120 or to the tissue. As discussed above, implant 110 may be secured to augment 120′ or augment construct 120 mechanically or adhesively.

As shown in FIGS. 1 and 2, the augment 120′ may be a patch. As is further shown, in a particular embodiment, the augment 120′ may be coupled to a substrate 122 to form the augment construct 120. The substrate 122 is coupled to the implant 110, and the augment 120′ is coupled to the substrate 122. In some aspects and as illustrated in FIG. 1, the augment 120′ is sutured to the substrate 122 and the substrate 122 is adhered to the implant 110. In other aspects, such as is illustrated in FIG. 2, the augment 120′ is adhered to the substrate 122 and the substrate 122 is adhered to the implant 110. However, the couplings between the implant 110, the augment 120′, and the substrate 122 are not limited to those aspects shown in FIGS. 1 and 2. For instance, the implant 110 and the augment 120′, and/or the substrate 122 and the implant 110, may be coupled to one another mechanically or chemically.

If it is included, the substrate 122 aids in coupling the augment 120′ to the implant 110. Since it is difficult to adhere the augment 120′ directly to the implant 110, the substrate 122 acts as an intermediary to couple the augment 120′ to the implant 110. For example, the substrate 122 may be adhered to each of the augment 120′ and the implant 110. The substrate 122 is optional. That is, the augment 120′ may be coupled directly to the implant 110 without use of the substrate 122.

According to other aspects of the present disclosure, which are not illustrated herein, the augment construct 120 is disposed within one or more walls of the implant 110 as an augment 120′ alone, without use of the substrate 122. In one example, the implant 110 is manufactured to include multiple surface layers, and the augment construct 120 or augment 120′ is manufactured interstitially between the layers. In another example, the augment construct 120 or augment 120′ is positioned inside implant 110. In still another example, the augment construct 120 or augment 120′ is constructed to be at least a portion of the structure of implant 110 itself, such that implant 110 and augment construct 120 or augment 120′ are a single structure. Prostheses 100 according to these aspects may not be assembled in situ by an end user, but rather may be premade.

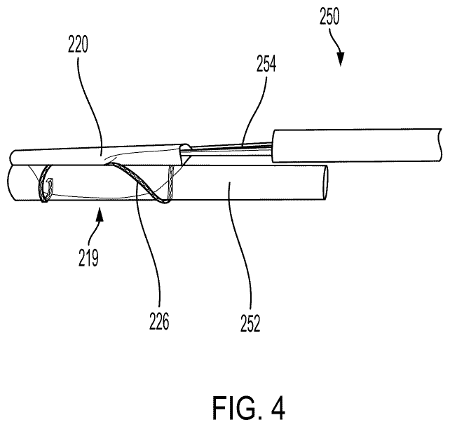

According to other aspects of the present disclosure, which are not illustrated herein, the prosthesis 100 may be used in tandem with other surgical procedures and/or implants as desired. For instance, continuing with the example herein of the repair of a rotator cuff, prosthesis 100 may be combined with a single row or double row rotator cuff repair, which are rotator cuff repairs well-known in the art. As a particular example, the suture(s) and/or suture anchor(s) may be used to create the single or double row repair (see e.g., paras. [0103-0112] and FIGS. 4-14 of U.S. application Ser. No. 17/825,569, such paragraphs and figures being incorporated by reference herein), and once this repair is performed, prosthesis 100 may be applied over top of the rotator cuff tissue and the repair suture(s) and suture anchor(s). In an alternative particular example, prosthesis 100 may be applied to the rotator cuff tissue first (or positioned underneath the tissue), and then the single or double row repair may be performed over top of prosthesis 100.

The augment 120′ may include a fibrous material which may include, for example, at least one of collagen, human collagen, bovine collagen, xenograph collagen, a synthetic material, a polyester material, an absorbable material, an organic material, silk, or combinations thereof. According to an aspect of the first embodiment, the augment 120′ is a collagenic material in the form of a compressed sheet. Alternatively, the augment 120′ may be a collagenic material in the form of an uncompressed sheet, in powder form, or in a fibrillar form which may be dispersed in a saline solution. Furthermore, uncompressed or compressed sheets can have variable density and/or thickness depending on the specific application. Collagenic materials typically degrade quickly in aqueous or acidic solutions, losing their predetermined form and clumping almost immediately after introduction to water. Further, acids in the human body can degrade collagen. However, a compressed collagenic sheet has a relatively long life before degradation in aqueous solutions such as saline as compared to other forms of collagen. For example, the compressed collagenic sheet may degrade after at least 6 months in an aqueous solution. Thus, a compressed collagenic sheet may be capable of longer use in the human body before biodegradation as compared to other forms of collagen.

The implant 110 may include a crystalline or semi-crystalline polymer, such as at least one of polycaprolactone (“PCL”), polycarbonate polyurethane (“PCU”), polyglycolide (“PGA”), polyhydroxybutyrate (“PHB”), plastarch material, polyetheretherketone (“PEEK”), zein, polylactic acid (“PLA”), polydioxanone (“PDO”), poly(L-lactic acid) (“PLLA”), poly(D-lactic acid) (“PDLA”), poly (DL-lactic acid) (“PDLLA”), 3:1 poly(L-lactide-co-E-caprolactone), poly(lactic-co-glycolic acid) (“PLGA”), and/or any other polymer suitable for rolling and inflation of the implant 110 as described herein. Implants 110 comprising such polymers generally remain inflated for a certain time period, such as for example about eight weeks, before breaches start to form as partial biodegradation occurs. These breaches result in the collapse of the implant 110 due to fluid leakage. Use of a hydrogel composition in the implant 110 may prevent or slow the leakage through the breaches, resulting in an extension of implant 110 inflation, such as for additional weeks or months. The implant 110 and the augment construct 120 or augment 120′ may each independently be biodegradable. For instance, the implant 110 and the augment construct 120 or augment 120′ may each be configured to biodegrade within 12 months following implantation into a patient.

In some aspects, the implant 110 has a volume of between 1 and 300 ml in the inflated configuration, such as between 9 and 11 ml, between 14 and 16 ml, between 23 and 26 ml, or between 50 and 60 ml. In some aspects, the implant 110 has an average wall thickness of between 25 and 400 microns, e.g., 100 microns.

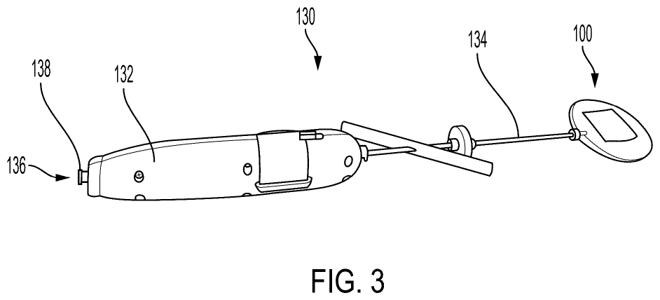

Further provided according to the first embodiment, and illustrated in FIG. 3, is a plurality of exemplary apparatuses including various prostheses 100 and implant delivery tools 130. In the insertion configuration, the prosthesis 100 is disposed on the implant delivery tool 130. The implant delivery tool 130 includes a handle 132 disposed at a proximal end of the implant delivery tool 130 and configured to be grasped by a user. An implant rod 134 extends from the handle toward a distal end of the implant delivery tool 130. The implant rod 134 typically has a length of at least 4 cm. A fluid path 136 runs throughout the implant delivery tool 130 from the proximal end to the distal end, through the handle 132 and implant rod 134. An injection port 138 is located at the proximal end and is in fluidic communication with the fluid path 136. The implant rod 134 is coupled to the prosthesis 100, which allows a user to inject a fluid through the injection port 138 and fluid path 136 and into the prosthesis 100. Such an implant delivery tool 130 is similar to or the same as the delivery tool associated with the aforementioned InSpace® balloon implant.

Further provided according to the first embodiment is an implantation method for implanting the prosthesis 100 within an internal space of a patient. The method includes providing the prosthesis 100 in the insertion configuration, the prosthesis 100 removably disposed on the implant delivery tool 130. The method further includes inserting the prosthesis 100 into a location within an internal space. In this step, the handle 132 is positioned by the user such that the implant rod 134 is positioned adjacent to the desired implantation location within the internal space. Thus, the user manipulates the implant delivery tool 130 via the handle 132 so that the prosthesis 100 is positioned appropriately within the internal space. The prosthesis 100 is arthroscopically inserted into the internal space while not inflated. After inserting the prosthesis 100, the method further includes inflating the prosthesis 100 to provide the prosthesis 100 in the implanted configuration. In this step the fluid is injected, for instance with a syringe, through the injection port 138, fluid path 136, and plug 112 and into the implant 110. The prosthesis 100 is configured to detach from the implant rod 134 once inflated. After inflation, the method further includes retracting the implant rod 134 from the position adjacent to the location within the internal space. The prosthesis 100 remains within the internal space in its inflated condition, thus delivering the implant 110 and augment construct 120 or augment 120′ to the target area within the internal space which may provide pain relief and joint function restoration.

Further provided according to the first embodiment is a method of manufacturing the apparatus including the prosthesis 100 (including the implant 110 and the augment construct 120 or augment 120′) and the implant delivery tool 130. A method of manufacturing the implant 110 combines dip molding and lost wax casting methods which are known in the art. Such exemplary techniques are described in U.S. Pat. No. 8,221,442, the entirety of which is incorporated by reference herein. Dip molding may be used to build the walls of the implant 110 by dipping a pre-shaped model of the implant 110 in a solution made of a polymer dissolved in an organic solvent. Suitable organic solvents include butanol, dichloromethane, chloroform, butanone, acetone, acetonitrile, disiopropyl ether, tetrahydrofurane, dioxane, ethyl and butyl acetate, and toluene. Suitable casting agents are hydrophilic in nature and include protein, polysaccharides and various synthetic and semisynthetic polymers. Examples of suitable casting agents include, but are not limited to, gelatin, agar, alginate, hydroxypropylcellulose, poly(acrylic acid-co-methylmethacrylate), chitosan, dextran, and arabinogalactane. Alternatively, alloys with a low melting temperature (e.g., alloys including rare earth metals) can be used for the casts. These casts are heated and melted and extracted at a temperature lower than the melting temperature of the coating polymer.

The manufacturing method may include steps in which a hot casting agent, such as 10% W/V agar in water, is prepared in an agitator at about 100 rpm and about 97° C. A mold is also prepared having the required implant shape. Then, the hot casting agent is injected into the mold. The cast is cooled to 30-35° C., then cooled again to 19° C. to solidify the cast. After cooling, an implant model is extracted from the mold and incubated for up to 10 days. The implant model is then dipped inside the dipping solution at a constant speed, such as about 20 cm/min, in one or more dipping and drying cycles (i.e., 24 hours drying at room temperature in sealed drying chamber). The dipping solution can be 10% W/V biodegradable polymer dissolved in an organic solvent. These steps can be repeated several times, for instance about six times, until the required coating thickness of the implant 110 is formed. The casting agent is then removed through pressing using combinations of automated and manual rollers. The implant 110 is then washed and dried, for instance with 50° C. water and drying for 30-60 minutes on a dryer.

When the implant 110 is filled with a biodegradable fiber, it can be alternatively fabricated by welding or gluing together two films of the implant material. Pressure forming, film extrusion or blown film methods are used to prepare the films. The films are then welded along the implant surface using an accurate and controlled ultrasonic energy or glued using an accurate deposit of organic solvent along the gluing path.

The manufacturing method according to the first embodiment further includes coupling the augment construct 120 or augment 120′ to the implant 110 to form the prosthesis 100. As described above, the augment construct 120 or augment 120′ and the implant 110 may be mechanically or chemically coupled to one another. In a particular aspect of the first embodiment, the manufacturing method includes wetting the augment construct 120 or augment 120′, puncturing the augment construct 120 or augment 120′ with a filament 126, coupling the implant 110 to the augment construct 120 or augment 120′ with the filament 126, drying the augment construct 120 or augment 120′, and packaging the formed and dried prosthesis 100. While the wetting and drying steps are optional, wetting the augment construct 120 or augment 120′ facilitates its puncture if a collagenic material is used, since such materials are typically tough and puncture-resistant when dried, but when wetted can be pierced easily. In an alternative aspect of the first embodiment, the manufacturing method includes puncturing the augment construct 120 or augment 120′ with a filament 126, coupling the implant 110 to the augment construct 120 or augment 120′ with the filament 126, and packaging the formed prosthesis 100, without wetting and subsequently drying the augment construct 120 or augment 120′.

The manufacturing method according to any embodiment herein includes manufacturing the implant delivery tool 130. Components such as the handle 132, the implant rod 134 having the fluid path 136, and the injection port 138 may be separately manufactured. The handle 132 may include two plastic pieces configured to join together around the implant rod 134. The handle 132 and implant rod 134 are manufactured to have a fluid path 136 running throughout the handle 132 and rod 134 from one end of the implant delivery tool 130 to the other. The injection port 138 may be attached to the implant rod 134 via a manufacturing process or manually by hand. Once the implant rod 134 and injection port 138 are provided, an injection molding process is carried out to form a flexible plastic rod sleeve (not shown in figures) surrounding the rod 134. The tip of the rod sleeve, which forms part of the inflation plug 112, is prepared by overmolding and then trimming with a surgical blade. Then, all remaining components of the implant delivery tool 130 including the handle 132 and implant rod 134 are manually assembled by a user to form the implant delivery tool 130. The manual assembly is carried out in a clean room to prevent any bacterial or other contamination.

According to the first embodiment, the manufacturing method further includes coupling the implant 110 to the implant delivery tool 130. First, the implant 110 is partially inflated. Then, dichloromethane is applied to the plug 112 and to the rod sleeve. The dichloromethane facilitates the attachment of the implant 110 to the implant delivery tool 130 and can be removed easily by evaporation. The rod sleeve and implant 110 are attached, and then the rod sleeve and implant 110 are clamped and cured at the inflation port 112, thus fusing the rod sleeve to the implant 110. Curing may be performed, for example, at about 90° C., or at about 90° C. or less, or at about 100° C. or less, or at about 110° C. or less. Then the assembled implant 110 is folded and, along with implant delivery tool 130, is packaged.

Provided in a second embodiment is another exemplary configuration of an augment and an implant, wherein the augment and the implant are not attached to one another prior to delivery to a surgical operator, but rather are attachable to one another to form the prosthesis either just prior to implantation or upon implantation within the patient. Furthermore, the prosthesis may be disposable on the implant rod of the implant delivery tool to form the apparatus, or may already be disposed on the implant rod. Indeed, in this embodiment, a standard InSpace® balloon implant may serve as the implant, which is later attached to an augment once within the patient or immediately therebefore through surgical methods described below. According to such an embodiment, the implant and augment can be assembled by an end user such as a surgical operator. For example, the user can wind the augment around the deflated implant to form the prosthesis in the insertion configuration. In another example, the augment and implant can be coupled to one another after being implanted, as described further below. Certain aspects of the aforementioned surgical methods relating to the first embodiment may be used in this exemplary embodiment as well, such as the implantation method steps described above where the surgical operator can implant and inflate the prosthesis, followed by detachment and removal of the implant delivery tool.

Further provided according to the second embodiment is a system which includes the prosthesis, the implant delivery tool, and an augment delivery tool. While these prostheses and tools may be presented to an operator as a single kit packaged in one or more packages, the operator may instead utilize an existing InSpace® balloon implant and delivery tool along with the new augment delivery tool described below for use in the new surgical methods also described below. One embodiment of an augment delivery tool 250 is shown in FIG. 4 and includes a plastic augment sleeve 252 configured to encapsulate the implant and implant insertion tool rod (not shown in FIG. 4, described below). The augment delivery tool 250 further includes an augment rod 254 disposed adjacent to (e.g., parallel to, as illustrated) the augment sleeve 252 and configured to position an augment construct 219 including an augment 220 proximate to the distal end of the implant delivery tool 230 and implant 210 (see FIG. 6) during implantation into a patient. In the embodiments depicted in FIGS. 4-8 and 31, the augment rod 254 includes a pair of grasping arms. However, it should be appreciated that such augment rod 254 is only one example of an augment rod, and the augment rod may also be a clamp, a clip and/or any other type of tool configured for removable coupling to the augment 220. The augment construct 219 further includes filaments 226 coupled to augment 220 and, as illustrated, encompassing the augment sleeve 252. As described later, such as relative to FIG. 6, when the augment 220 is coupled to the implant, the filaments 226 may serve as the coupling element.

Further provided according to the second embodiment is a kit. The kit includes the system of the second embodiment and further includes sterile packaging in which the implant 210 and the augment construct 219, and associated tools, are removably packaged in a sterile state.

Further provided according to the second embodiment is a method of manufacturing the implant 210, which is similar in most respects to that described above in the first embodiment and implant 110.

The manufacturing method according to the second embodiment differs from that of the first embodiment in that it excludes the step of coupling the implant 210 to the augment 220. Instead, the implant 210 and augment 220 are coupled to one another by a surgeon or surgical assistant after the implant 210 and augment 220 have been separately manufactured. The coupling can be performed before, during, or after the implant 210 and the augment 220 are implanted into a patient.

According to the manufacturing method of the second embodiment, the implant 210 may be coupled to the implant delivery tool 230 as described above in the first embodiment. Alternatively, the implant 210 and implant delivery tool 230 may be manufactured and packaged separately.

The manufacturing method according to the second embodiment includes attaching the filaments 226 (which may be sutures according to one or more aspects of the present disclosure) to the augment 220. The augment 220 is first optionally wetted to allow it to be more easily punctured with a suture needle. After the optional wetting, the augment 220 is pierced with filaments 226 which extend outward from the augment with a sufficient length to allow the filaments 226 to either pierce or tether the implant 210. Then, if wetting was performed, the collagen is dried before packaging the augment 220. In the second embodiment the augment 220 is so prepared during manufacture.

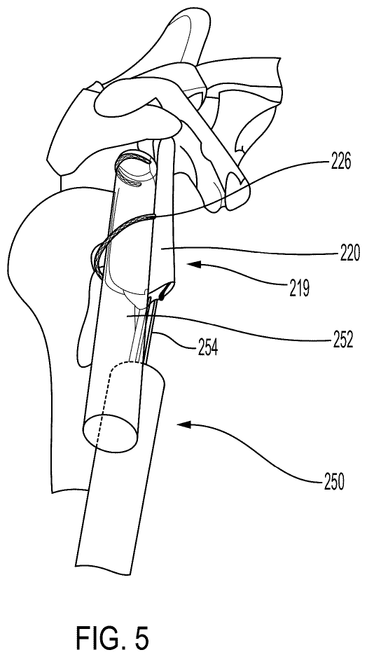

Further provided according to the second embodiment is an implantation method, and portions of such an exemplary method are illustrated in FIGS. 5-8. The method may be similar to that described above with respect to the first embodiment, but includes different and/or additional steps as described herein. For instance, the implantation method according to the second embodiment includes providing an implant 210 (e.g., a balloon implant) in an insertion configuration, such as disposed within a rod 234 of an implant delivery tool 230, providing an augment 220 in an insertion configuration and disposed on an augment delivery tool 250, and attaching the augment 220 to the implant 210 to form a prosthesis 200 in an insertion configuration. These steps may be performed in whole or in part before, during, or after the implantation method steps described above with respect to the first embodiment. Implant delivery tool 230 includes a sheath 240 positioned over rod 234, the sheath 240 being slidable along rod 234 between a retracted and an extended position. As with implant delivery tool 130, implant delivery tool 230 includes a fluid path (not shown) that runs throughout the implant delivery tool 230 from the proximal end to the distal end, through the handle and implant rod 234. An injection port is located at the proximal end and is in fluidic communication with the fluid path.

The implantation method of the second embodiment may enable a smaller incision than that of the first embodiment, since the assembled prosthesis 100 of the first embodiment occupies a larger diameter than when disassembled into the component implant 210 and augment 220 as presented here. For example, the incision size needed for the implant 210 alone may be less than 8 mm, or less than 5 mm. The second embodiment is therefore advantageous compared to the first embodiment in that a smaller diameter incision may be made into the skin of the patient. The first embodiment is advantageous in that its implantation method is simplified; since the prosthesis 100 is already assembled, no assembly is required by the surgeon before implantation.

However, requiring the surgeon to assemble the prosthesis 200, as set forth below, provides an additional advantage of flexibility. For instance, a surgeon could use auto/allograph tissue, or other non-collagenic material for the augment. Live tissue could not be used to manufacture the augment since such live tissue would decay in storage. Thus, forming the prosthesis 200 immediately before, during, or after implantation allows for greater flexibility in augment materials. Still further, as noted above, the standard InSpace® balloon implant 210 and inserter can be used in this exemplary method. Moreover, for this second embodiment, separating the packaging of the implant 210 from the packaging of the augment 220 may be advantageous for different manufacturing or packaging requirements between the components such as one needing refrigeration or freezing, regulatory restrictions such as being approved as a combination device instead of a standalone device, bonding requirements such as the adhesive needing to be in the presence of the patient's autologous blood, PRP (Platelet Rich Plasma), BMAC (Bone Marrow Aspirate Concentrate) or other autologous substance, or an enhanced efficacy potential where one component should be in the presence of blood, PRP, BMAC, or other substance separated and/or prior to combining the implant 210 and augment 220 for implantation.

FIGS. 5-8 illustrate particular aspects of the implantation method according to the second embodiment, wherein the implant 210 and augment 220 may be coupled via filaments 226 (shown as sutures in FIGS. 5-8) before, during, or after being implanted. As shown in each of FIGS. 5-8, the implant 210 and augment 220 can be attached via filaments 226 by positioning implant 210 relative to augment 220 at some point during the implantation method. As a result of this positioning, the prosthesis 200 is deployed within the internal space. In an aspect of the present disclosure, the filaments 226 are guided through the implant 210 to couple augment 220 and implant 210 during implantation. According to such an aspect, the filaments 226 are in contact with or encompass the sleeve 252 such that the implant 210 unfolds into the filament arrangement upon inflation, placing the augment 220 proximate to the target tissue. In an alternative aspect, the filaments 226 couple the augment 220 and implant 210 upon inflation of implant 210. As specifically illustrated in FIG. 6, the surgeon arranges the implant 210, augment 220, and filaments 226 such that upon either implantation or inflation of implant 210, filaments 226 couple augment 220 to the surface of implant 210, similar to certain examples of the first embodiment above. Further, since augment 220 is retained to implant 210 through filaments 226, a substrate (e.g., substrate 122 as discussed above) may not be included, though it still can be if desired.

FIG. 5 further illustrates a portion of the step of inserting the prosthesis 200 into a location within an internal space according to the second embodiment. Specifically, FIG. 5 shows insertion by a user of the augment delivery tool 250 into the internal space. In FIG. 5, the augment 220, filaments 226, augment delivery tool 250, and sleeve 252 are shown without the accompanying implant delivery tool 230 and implant 210 (and thus the assembled prosthesis 200 is not shown). The augment sleeve 252 may be positioned through an incision on a patient, such that when the deflated implant 210 is inserted through the augment sleeve 252, the implant 210 can pass through the incision and into the internal space. Further, since sleeve 252 is positioned within filaments 226, the eventual insertion of implant 210 through sleeve 252 may result in positioning the filaments 226 within the implant 210 as well. As illustrated in FIG. 5, augment 220 is positioned within the patient anatomy where eventual implantation is intended. Though, augment 220 in this step could be positioned elsewhere in the anatomy, such as within the patient but adjacent to the intended implantation site.

FIG. 6 shows such insertion of the implant delivery tool 230 into the augment sleeve 252 such that implant 210 is now positioned within both sleeve 252 and filaments 226. As with FIG. 5, this combination of implant 210 to augment 220 could occur at or adjacent to the intended implantation site. Of course, these steps of FIGS. 5 and 6 could also occur outside of the patient, though performing the method in this manner may inhibit the benefit of this second embodiment of allowing for a reduced incision through the patient's soft tissue to access the joint and eventual implantation site.

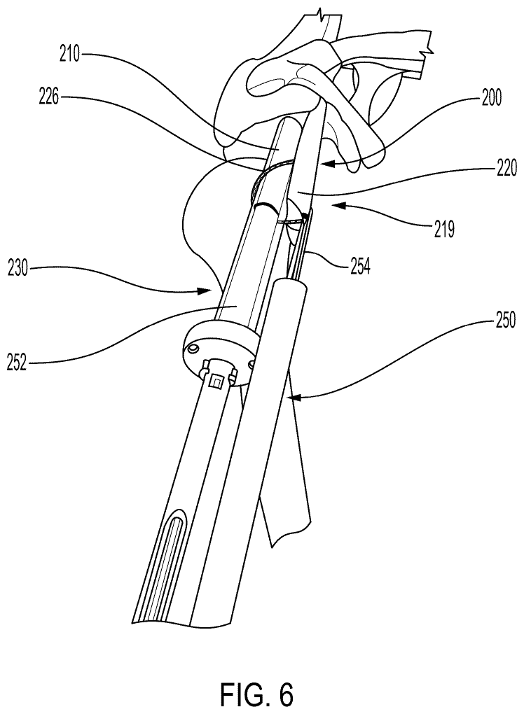

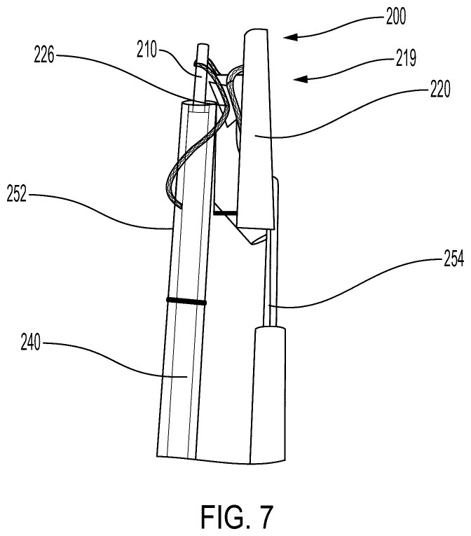

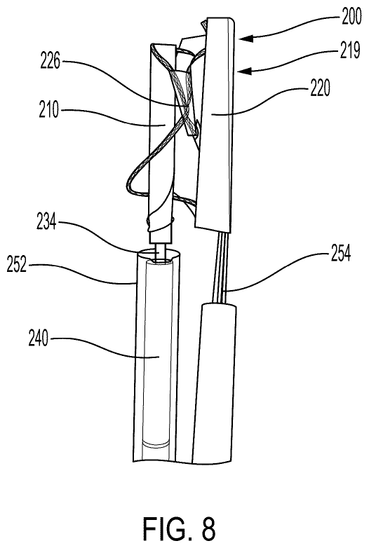

FIGS. 7 and 8 illustrate the step of retracting the implant rod 234 from the position adjacent to the location within the internal space (though of course these figures are only representative and are shown as being outside of the patient). FIG. 7 shows the system before retraction. FIG. 8 shows the system after retraction of the augment sleeve 252 and the implant rod 234, which leaves the prosthesis 200 remaining in the internal space and ready for inflation of implant 210. As the augment sleeve 252 and implant rod 234 are retracted, the filaments 226 and the augment rod 254 hold the augment 220 in place adjacent to the implant 210.

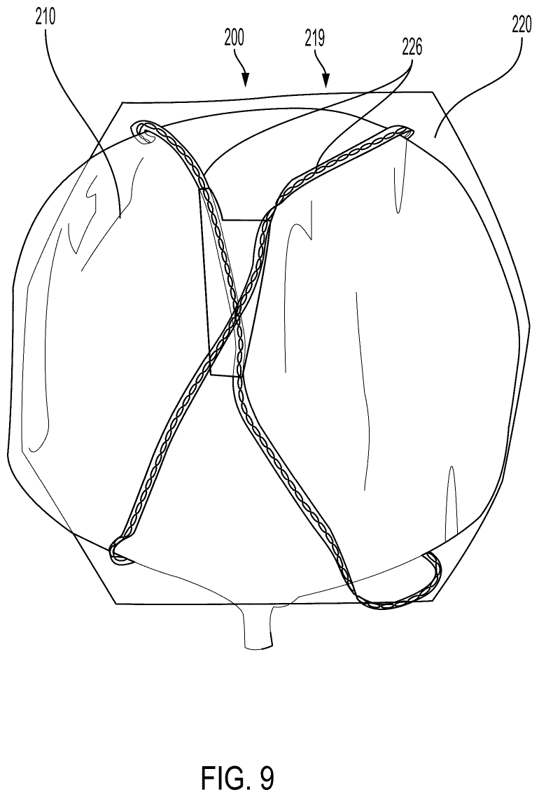

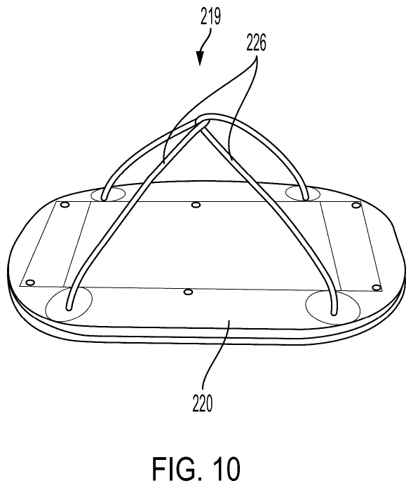

FIG. 9 shows the inflated implant 210 and the completed prosthesis 200 as would be positioned at the intended implantation site. Though the prosthesis 200 is not shown disposed within an internal space, according to the method described herein, the prosthesis 200 is inflated after being formed and implanted in the internal space. As illustrated more clearly in FIG. 10 and according to a particular aspect of the second embodiment, there is provided a plurality of filaments 226 configured to secure the implant 210 to augment 220 in the implanted configuration. As discussed above, the augment 220 and filaments 226 may constitute an augment construct 219.

The desired implantation site may be on top of rotator cuff tissue (if present) and the humeral head and below the acromion. As with the InSpace® balloon implant, the prostheses 200 of the present disclosure are intended, for instance, to restore the subacromial space in a patient's shoulder joint, while the augment 220 is intended to stimulate soft tissue regrowth and healing. While this prosthesis 200 of the present disclosure can be used with so-called massive rotator cuff tears that cannot be repaired by other means (e.g., by the use of sutures and suture anchor, or other surgical rotator cuff repair), it may be used in combination with such other known repair techniques such as over a standard rotator cuff repair. The prosthesis 200 is implanted arthroscopically, though an open procedure may be used if desired.

Further as to this second embodiment, as illustrated most clearly in FIGS. 9 and 10, filaments 226 may intersect one another or otherwise form a filamentary web to capture the implant 210 within the filaments 226. For example, the augment construct 219 may include the augment 220 and the filaments 226. The space between the augment 220 and the filaments 226 may define a receptacle configured to receive the implant 210. In some examples, the receptacle may receive the implant 210 in a deflated condition and may restrain movement of the implant 210 after the implant 210 is inflated. For example, when the implant 210 is inflated, it presses against the filaments 226, making the filaments 226 taut and holding the implant 210 in place against the augment 220. FIG. 10 shows a similar construct as FIG. 9, but without the implant 210. Though only two lengths of filament 226 are shown in FIGS. 9 and 10, the present disclosure is not limited thereto. For instance, a single filament 226 having one or more segments, or multiple individual filaments 226, may be used to create a filamentary web with higher or lower contact area to secure the implant 210 and augment 220. In some embodiments, the augment 220 may be coupled (e.g., adhered, stitched, etc.) to another structure (e.g., a substrate similar to substrate 122) that may be coupled to the filaments 226 rather than coupling the augment 220 directly to the filaments 226.

According to a particular aspect of the second embodiment, the augment 220 is attached to the implant 210 by a surgeon mechanically with filaments 226 (which may be sutures according to particular aspects of the present disclosure). The augment construct 219 is pre-made with associated filaments 226 coupled to the augment 220, as described herein as to the manufacturing method of the second embodiment. The filaments 226 extend outward from the augment 220 and are capable of coupling the implant 210 to the augment 220, preferably by tethering a portion or all of the implant 210, though in some variations one or more filaments 226 could also pierce or otherwise be adhered to the implant 210. In another particular aspect of the second embodiment, the surgeon manipulates the filaments 226 to couple the implant 210 and augment 220 directly to one another after implantation. First, the implant 210 is inserted with the implant delivery tool 230 and the augment 220 is separately inserted adjacent to the implant 210 with the augment delivery tool 250 either before or after the implant 210. Then, the implant 210 and the augment 220 are attached with the filaments 226 (e.g., by stitching through the implant 210 and securing the filaments 226 to the augment 220) or secured with the filaments (e.g., by passing the filaments 226 around the implant 210 and securing the filaments 226 to the augment 220) in the internal space by the surgeon. In another alternative aspect, the augment 220 is attached to the implant 210 by a surgeon using a substrate. The substrate is stitched to the augment 220 and the substrate is adhered to the implant 210. Thus, the substrate acts as an intermediary, since it may be difficult to either adhere or stitch the augment 220 and implant 210 directly to one another. In either case, the surgeon can partially inflate the implant 210 to couple the implant 210 and augment 220. Alternatively, the surgeon could couple the implant 210 and augment 220 without inflating the implant 210. Inflating the implant 210 at least partially may provide a more robust surface, facilitating the attachment.

Provided in a third embodiment is an apparatus including the prosthesis, the implant delivery tool, and the augment delivery tool. The prosthesis, the system, the kit, the manufacturing method, and the implantation method according to the third embodiment are similar to the second embodiment, except that, for instance, the augment and associated sutures are not pre-formed, such as during manufacture, but instead the operator assembles the suture(s) and augment for association with the implant. Specifically, the operator performs the steps of stitching the suture(s) to the augment, and alternatively or in conjunction, stitching the sutures around the implant prior to or following inflation of the implant.

The manufacturing method according to the third embodiment, like that of the second embodiment, may exclude the step of coupling the implant to the augment. Instead, according to the third embodiment, the implant and augment are coupled to one another after the implant and augment have been separately manufactured, as part of the implantation method (i.e., before, during, or after being implanted by a surgeon). In a particular aspect, the implant and augment are coupled after being implanted into a patient. For example, after implanting the implant and augment separately, the surgeon couples the augment to the implant using the filaments, similarly to what was described above with respect to the second embodiment.

According to an aspect of the third embodiment, the implantation method includes the step of attaching the augment to the implant mechanically with filaments, similar to aspects of the second embodiment. Unlike such aspects of the second embodiment, however, the implantation method according to this aspect of the third embodiment includes a step of stitching the filaments to the augment (before attaching the augment and implant to one another with said sutures). In a particular aspect, the augment is wetted to allow for easier puncture, and then a surgeon stitches the filaments through the augment. In an alternative aspect, the surgeon stitches the filaments through the augment in a dry state, without first wetting the augment. The third embodiment differs from the second embodiment in that the augment is (optionally wetted and) pierced with sutures by the surgeon, not by the manufacturer. The implantation method according to an aspect of the third embodiment therefore requires the additional step of stitching the sutures to the augment, before coupling the augment and implant together. Thus, the third embodiment provides a simpler manufacturing process, but a more complex implantation process as compared to the second embodiment, since the surgeon must prepare the augment with sutures before coupling to the implant.

Notably, in each of the second and third embodiments, there is provided an augment delivery tool for coupling the augment to the implant to form the prosthesis during insertion of the prosthesis inside the internal space. By contrast, in the first embodiment, as shown in FIG. 3, the augment is coupled to the implant without use of an augment delivery tool, and the prosthesis is inserted only with use of the implant delivery tool. Since in the first embodiment the implant and augment are pre-assembled into the prosthesis, the first embodiment requires only a single delivery tool to implant the formed prosthesis. Thus, the manufacturing process is simplified and material requirements are reduced according to the first embodiment as compared to the second and third embodiments. Conversely, the second and third embodiments provide for more compact instruments and devices during insertion into a patient and provide for greater flexibility to the surgeon, and to packaging of the various tools and devices.

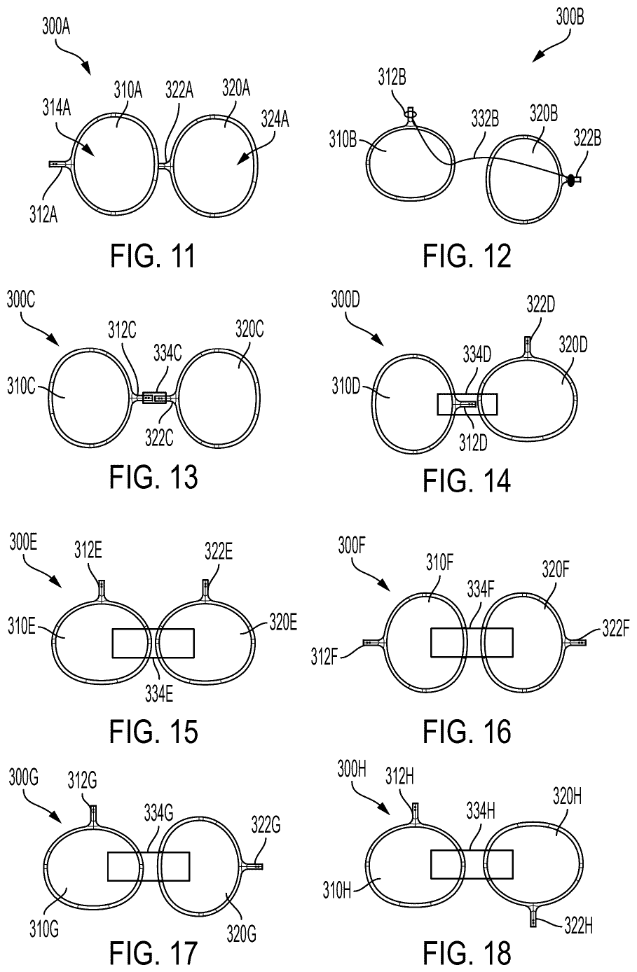

In some embodiments, a prosthesis includes a dual implant and may optionally include a receiver assembly, an augment, or a receiver assembly and an augment. We begin with a description of the dual implant of the prosthesis. Such dual implant may be used for a variety of purposes. For instance, part of a dual implant may be positioned within a glenohumeral joint of a patient to relieve complications resulting from arthritis with another part positioned to improve the patient's passive range of motion in the joint. In other instances, the dual implant may be included as part of a solution to treat a significant rotator cuff tear. Additionally, the dual implant may also provide stability in its implanted location such that it may be expected to reliably remain in the implanted location without fixation. One variation of the dual implant is a dual balloon. Examples of dual implant prostheses are shown in FIGS. 11-19. In FIGS. 11-19, each implant of the dual implant is a balloon-type dual implant.

Details of prosthesis 300A illustrated in FIG. 11 will now be described. It should be appreciated that prosthesis 300A is representative of the prostheses 300A-300I unless otherwise noted. Prosthesis 300A includes a first implant 310A and a second implant 320A. Implant materials for prosthesis 300A may be as described for implant 110. Each of first and second implants 310A, 320A includes a respective interior volume 314A, 324A that is variable as a function of an extent to which each implant 310A, 320A is inflated. An inflatable volume of each implant 310A, 320A may be as described for implant 110. Further, each implant includes an inflation port, first inflation portion 312A for first implant 310A and second inflation port 322A for second implant 320A. In prosthesis 300A, second inflation port 322A is directly attached to first implant 310A, and first inflation port 312A extends from a side of first implant 310A opposite the attachment to second inflation portion 322A, as shown in FIG. 11. Attachment between first and second implants 310A, 320A via second inflation port 322A may be through various means. For example, second inflation port 322A may be subject to heat treatment to melt it onto first implant 310A. In other examples, a weld or an adhesive may be used to attach second inflation port 322A to first implant 310A. In any of the above examples, first implant 310A may be prepared so that once attached, first and second implant 310A, 320A are in fluid communication with each other. In still further examples, first implant 310A and second implant 320A may be formed monolithically as a single structure where the interior volumes 314A, 324A are in fluid communication with each other. In prosthesis 300A, inflation port 312A functions as an inlet for both first implant 310A and second implant 320A. To the extent not explicitly mentioned above, any characteristics contemplated for implant 110 may also be provided for first and second implants 310A, 320A.

Turning to the prostheses illustrated in FIGS. 12-19, prosthesis 300B in FIG. 12 includes a filament 332B having a first end attached to first inflation port 312B of first implant 310B and a second end attached to a second inflation port 322B of second implant 320B. Such attachment of filament 332B may be through tying to the respective inflation ports. In one example placement within a patient, the respective ports are oriented so that first inflation port 312B is offset from an axis through the centers of both first and second implants 310B, 320B, while second inflation port 322B may be positioned approximately along the axis. In other examples, the first and second inflation ports 312B, 322B may be oriented in other directions as desired.

Prosthesis 300C is illustrated in FIG. 13 and includes first implant 310C and second implant 320C attached via a supplemental material segment 334C that may be applied to the respective implants with an adhesive. In some examples, respective first and second inflation ports 312C, 322C may be bridged by supplemental material segment 334C. In other examples, an adhesive may be used to directly bond the first and second implants 310C, 320C together. As to materials used, in some examples, supplemental material segment 334C may be made of the same material as first and second implants 310C, 320C.

Prostheses 300D-300H are illustrated in FIGS. 14-18. Unless otherwise indicated, like reference numerals refer to like elements of prosthesis 300C shown in FIG. 13, but within the respective 300D, 300E, 300F, 300G and 300H-series of numerals. For prosthesis 300D, supplemental material segment 334D attaches to first implant 310D and second implant 320D. Attachment to first implant 310D may be via first inflation port 312D. Second inflation port 322D is spaced apart from the attachment location and is oriented approximately orthogonally relative to a centerline axis through first and second implants 310D, 320D. For prosthesis 300E, supplemental material segment 334E attaches to first implant 310E and second implant 320E. Each of first and second inflation ports 312E, 322E are spaced apart from supplemental material segment 334E and are oriented in a direction orthogonal to a centerline axis through the first and second implants 310E, 320E. For prosthesis 300F, supplemental material segment 334F attaches to first implant 310F and second implant 320F. First and second inflation ports 312F, 322F are spaced apart from the attachment location between the implants but are aligned with a centerline axis through both implants 310F, 320F. For prosthesis 300G, supplemental material segment 334G attaches to first implant 310G and second implant 320G. First inflation port 312G is oriented approximately orthogonally to and spaced apart from a centerline axis between the implants 310G, 320G, while second inflation port 322G is parallel to the centerline axis and spaced apart from supplemental material segment 334G. For prosthesis 300H, supplemental material segment 334H attaches to first implant 310H and second implant 320H. First inflation port 312H and second inflation port 322H are oriented approximately orthogonal to and are both offset from a centerline axis extending across first and second implants 310H, 320H, with first inflation port 312H being on a first side of the centerline axis and second inflation port 322H being on a second side of the centerline axis.

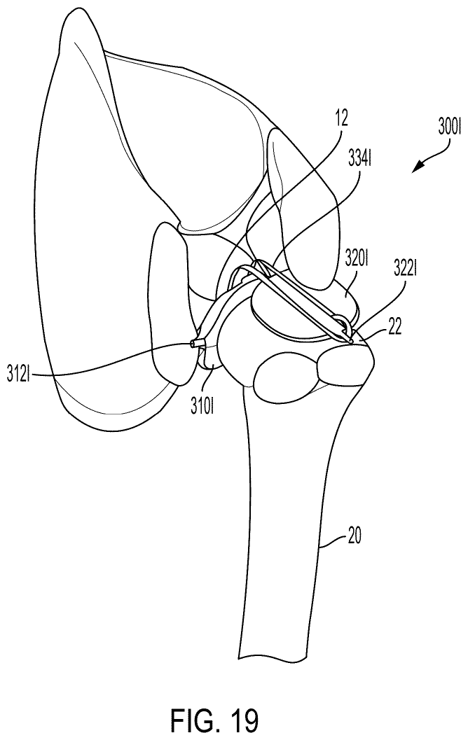

Prosthesis 300I is illustrated in FIG. 19 and is shown implanted in a shoulder joint of a patient. Unless otherwise indicated, like reference numerals refer to like elements of prosthesis 300C shown in FIG. 13, but within the 3001-series of numerals. Prosthesis 300I includes a first implant 310I, a second implant 3201 and a supplemental material segment 334I that attaches implants 310I, 320I together. As shown, supplemental material segment 334I includes a looped end at second inflation port 322I and two parallel sub-segments extending from the looped end, each of the parallel sub-segments being attached to first implant 310I. As described in greater detail elsewhere in the present disclosure, prosthesis 300I may be manipulated so that a relative orientation between first implant 310I and second implant 320I best suits an anatomical placement. Further, prosthesis 300I is designed so that when implanted in a patient, a position of the respective first and second implants 310I, 320I may be such that the prosthesis 300I is self-stabilizing and may be expected to stay in its implanted position without the need for separate fixation of either first or second implant 310I, 320I to tissue of the patient.

The dual implant prosthesis may be varied in many ways. In some examples, any one dual implant structure from among the contemplated dual implant structures may include a first implant with a first size and a second implant with a second size different from the first size. Such implant sizes may be customized to suit a specific space in which the implant is to be placed within a patient. For instance, a first implant of a dual implant may be sized for disposal in a subacromial space while a second implant of the dual implant may be sized for disposal within the intra-articular shoulder joint between the glenoid 12 and the head 22 of humerus 20, as shown in FIG. 19. Such applications may be advantageous when treatment seeks to solve more than one problem. In some embodiments, each implant of a dual implant may be configured to be filled and/or inflated separately, and in others, such as prosthesis 300A illustrated in FIG. 11, the implants may be filled and/or inflated together.

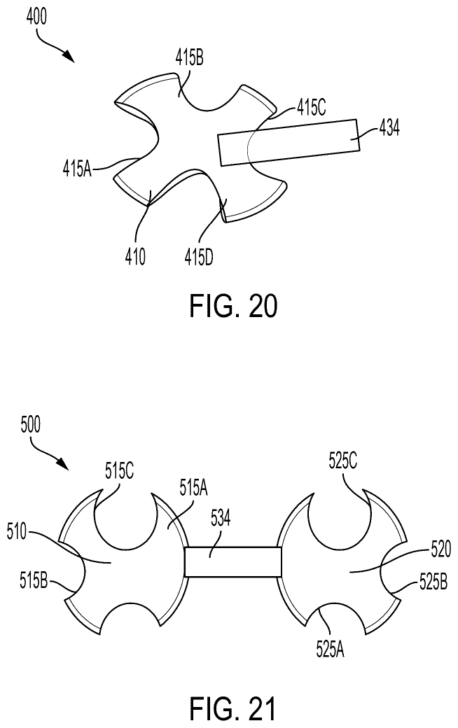

In some embodiments, and as mentioned above, a prosthesis may include an implant and a receiver assembly. A receiver assembly is a device that is sized to receive an implant and may be used in conjunction with a single implant or a dual implant. The receiver assembly has flexible material properties for case of manipulation and to provide versatility in its surgical applications. A receiver assembly 400 is illustrated in FIG. 20. Receiver assembly 400 may be made of the same materials as implant 110 or augment 120. In some examples, receiver assembly 400 may be made at least in part from collagen. In some examples, at least a portion of receiver assembly 400 may be an augment or may be coupled to an augment.

Receiver assembly 400 may have a body 410 made from a balloon-type structure with spaced-apart peripheral cutouts. Throughout the present disclosure, a body of a receiver assembly may also be referred to as a receiver body. As shown in FIG. 20, body 410 of receiver assembly 400 includes first, second, third and fourth peripheral portions 415A-D, each being spaced apart from the others by cutouts in body 410. While accessible from multiple directions, internal volume (cavity, etc.) of body 410 is also sized to receive an implant, e.g. implant 110. The internal volume of body 410 is expandable in conjunction with expansion of the implant from within body 410. Receiver assembly 400 may also optionally include a supplemental material segment 434 to modify receiver assembly 400 into a two body assembly such as that shown in FIG. 21, described in greater detail below.

The receiver assembly 400 may include an augment and may be referred to as an “augment construct.” For example, the body 410 of the receiver assembly 400 may be coupled to an augment 120 or may itself be an augment. The receiver assembly 400 may be inserted into an implantation site, and the body 410 may be affixed to the implantation site. An implant (e.g., the implant 110) may be positioned in the internal volume of the body 410 before or after insertion of the receiver assembly 400 into the implantation site. The materials of the implant 110 may biodegrade, for example, over a period of weeks, leaving the body 410 of the receiver assembly 400 empty at the implantation site. The body 410 may be made of the same materials as the augment 120, including collagen, cross-linked collagen, non-cross-linked collagen, reconstituted collagen, native collagen, human collagen, bovine collagen, xenograph collagen, a synthetic material, a polyester material, an absorbable material, an organic material, silk, or combinations thereof. Alternatively, a separate augment 120 may be coupled to the body 410. After the implant 110 biodegrades, the empty body 410 of the receiver assembly 400 may remain at the implantation site, acting as an augment or holding a separate augment 120 in place. In examples in which an augment is coupled to the body 410, the augment may be coupled using any of the mechanical or chemical methods discussed above for coupling the augment 120 to the implant 110, including wetting the augment 120 to adhere the augment 120 to the receiver body 410. The internal volume of the body 410 may be referred to as a cavity or receptacle and may be configured to receive an implant (e.g., implant 210) in a deflated condition and may restrain movement of the implant after the implant is inflated.

The balloon implant 110 may be inserted in a collapsed (e.g., folded, rolled, etc.) and deflated configuration into the internal volume of the body 410 through the cutouts formed between the peripheral portions 415A-D. When the balloon of implant 110 is inflated, positional movement of the balloon implant 110 is restrained by peripheral portions 415A-D, which hold the implant 110 in the internal cavity of the body 410. According to some aspects, a dimension of the balloon implant 110, when inflated, may be larger than an adjacent dimension of the cutout in order to positionally restrain the movement of the balloon implant 110, and the size of the inflated balloon implant may restrict the removal of the balloon implant from the cavity through any of the one or more cutouts. The cutouts are thus large enough to receive the implant 110 in a deflated, collapsed configuration but not so large as to allow the implant 110 to be removed from the internal volume when in an inflated, expanded configuration (e.g., without tearing the body 410 or using substantial force beyond what is expected during normal use of the joint in which the implant 110 is implanted). For example, in some embodiments, there is at least one point within the inflated implant wherein every cross-section of the implant passing through that point has a larger area than any of the cutouts, such that the inflated implant cannot fit through the cutouts in any orientation. For example, in the case of a spherical inflated implant with a larger diameter than a diameter of a circular cutout, every cross-section of the implant passing through the center of the sphere will have a larger area than the area of the cutout, and the implant will not fit through the cutout. In some embodiments, the inflated implant is larger in two perpendicular dimensions passing through at least one point within the inflated implant than a maximum dimension of the cutout. Thus, for example, if the implant is roughly disk shaped, with a diameter larger than a maximum dimension of the cutout (e.g., diagonally across a rectangular cutout), the length and width of the implant along lines passing through the center of the implant will be larger than the maximum dimension of the cutout (while the height of the implant may be smaller than the maximum dimension of the cutout). Thus, even if the cross sectional area of the implant is not larger than the cutout, the implant will still not fit through the cutout. In some embodiments, a portion of the implant 110 adjacent to the opening is larger (e.g., has a larger cross-sectional area than) than the cutout.

A receiver assembly 500 with two bodies 510, 520 is illustrated in FIG. 21. Unless otherwise indicated, like reference numerals refer to like elements of receiver assembly 400 shown in FIG. 20, but within the 500-series of numerals. Each body 510, 520 includes three cutouts defining respective peripheral portions 515A-C and 525A-C. First peripheral portions 515A, 525A on respective bodies 510, 520 are joined via a supplemental material segment 534. In use, receiver assembly 500 may be manipulated to change a position and orientation of body 510 relative to body 520 and vice versa. As with receiver assembly 400, each body 510, 520 includes an internal volume adapted to receive an implant and to expand with expansion of the implant. For each contemplated receiver assembly, it should be appreciated that when an implant is disposed within a body of a receiver assembly, any inflation port or other access on the implant may be positioned, e.g. rotated within the body, so that it is accessible through a desired cutout in the body. Receiver assemblies 400, 500 are also advantageous in that the cutouts in the material render it easier to roll up such receiver assemblies 400, 500 within a sheath of an implant delivery tool, such as implant delivery tool 230.

In variations, a receiver assembly may have any number of cutouts. For example, a receiver assembly may have a single cutout in the body, two cutouts, three cutouts, and so on. A size and quantity of cutouts may have a limit based on an extent of material that must remain to provide necessary strength to the receiver assembly. In this manner, a volume of material removed from a body of the receiver assembly may be limited to avoid an overly deleterious effect on strength properties of the receiver assembly. In some variations, a receiver assembly may be supplemented by or otherwise include the properties of an augment, such as augment 120. For example, a receiver assembly arranged in such manner may include a fibrous material that may stimulate soft tissue regrowth and healing.

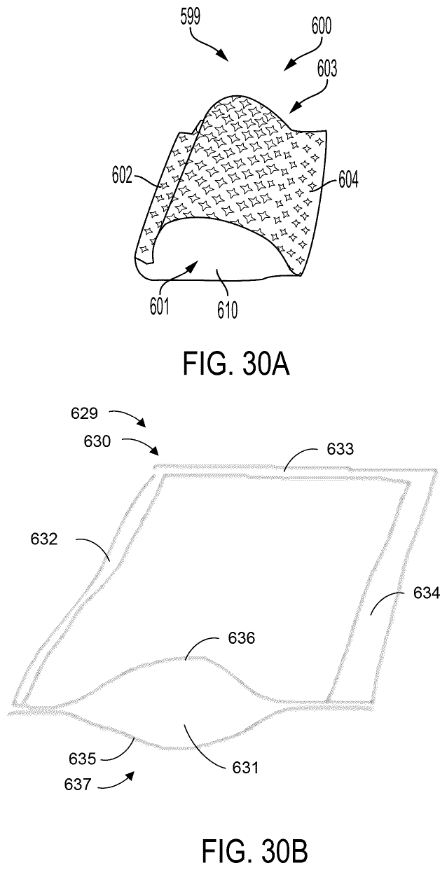

In some embodiments, and as mentioned above, a prosthesis may include an implant and an augment construct. One embodiment of such augment construct is augment construct 599, illustrated in FIG. 30A. As shown in FIG. 30A, augment construct 599 includes only augment 600. However, in other embodiments, augment construct 599 may include additional components. Augment construct 599 is configured to receive any implant, such as a single implant 110 or a dual implant 310, 320. Augment 600 defines a cavity 610 therein that is accessible from openings 601, 603 at opposite ends of augment 600. Closed edges of the augment 600 extending between the opposite openings 601, 603 include first edge 602 and second edge 604. Augment 600 may be used in various ways in a surgical procedure, described in greater detail elsewhere in the present disclosure. In some examples, augment 600 may be made of any of the materials contemplated for formation of augment 120. Additionally, augment 600 may include any of the characteristics contemplated for augment 120. For example, augment 600 may stimulate soft tissue regrowth and healing. As discussed above, augment construct 599 may include additional components in addition to an augment. For example, the augment construct 599 may include a receiver structure formed into a shape similar to that of augment 600 shown in FIG. 30A such that the receiver structure similarly defines a portion or portions of cavity 610, and the augment (which may be, for example, a patch similar to augment 120′) may be coupled to the receiver structure (e.g., to the bottom of the receiver structure). As another example, the augment construct 599 may include an augment that forms one side of the shape shown in FIG. 30A, and a receiver structure that forms the remainder of the shape, such that the augment and the receiver structure cooperate to define the cavity 610. Augment construct 599 may further include filaments (e.g., sutures) used to stitch edges 602, 604, which may optionally also define a portion or portions of cavity 610.

As shown in FIG. 30A, edges 602, 604 are continuously closed, but in some examples, either or both of edges 602, 604 may be non-continuously closed, with opening(s) 601, 603 providing access to cavity 610 (which may not be large enough for an inflated balloon to pass therethrough). In some examples, the openings 601, 603 at either end of augment 600 may be completely open from edge 602 to edge 604. In other aspects, the edges of the augment 600 forming the opening 601, 603 may be partially coupled, for example, adjacent the edges 602, 604 such that the opening 601, 603 does not extend fully from edge 602 to edge 604. In some examples, the edges surrounding at least one of the openings 601, 603 may be coupled (e.g., adhered, stitched, etc.) together after a balloon has been positioned within cavity 610 to close one or both of the openings 601, 603. In other examples, augment construct 599 may include a suture or filament coupled to openings or eyelets at the edges surrounding at least one of the openings 601, 603 that may act as a drawstring that may be tensioned to close the opening 601, 603 after a balloon has been inserted into cavity 610 (e.g., a purse string closure). FIGS. 41-42 illustrate some of these examples. In some examples, a suture or filament may be stitched through the augment 600 (or structure coupled to the augment 600) as opposed to being coupled to pre-existing openings or eyelets. For example, a suture or filament may encircle an opening 601, 603, passing through the material of the augment 600 several times along the edges of the respective opening 601, 603 before being tensioned to close the opening 601, 603 after a balloon has been inserted into cavity 610. Alternatively or in addition, the cavity 610 may have a varying cross-section size and/or shape extending between 601, 603, such that, similar to other examples above, at least one of the openings 601, 603 may be sized to allow a deflated implant to pass therethrough, but prevent egress of an inflated implant. In this instance, cavity 610 may have a larger cross-sectional shape at one or more locations in between 601, 603.

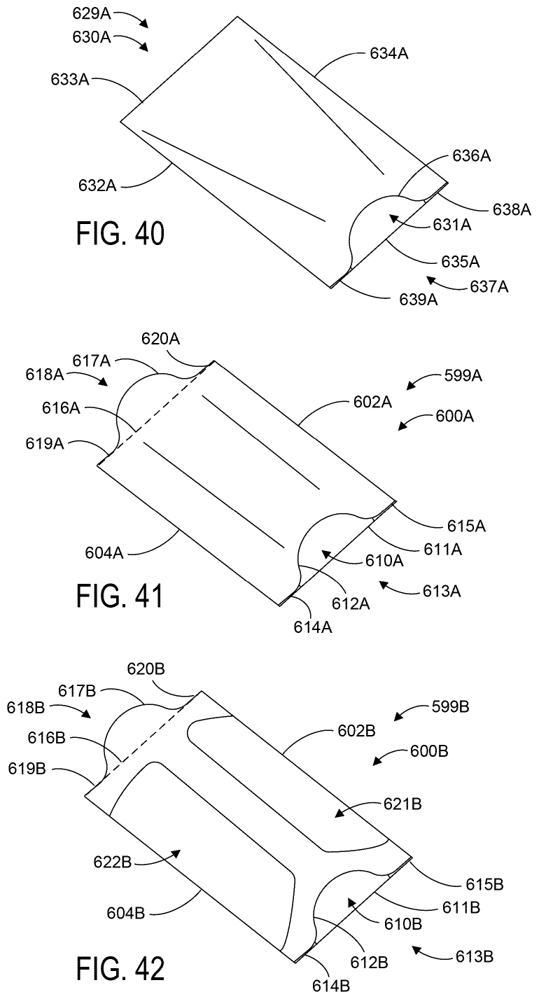

Referring now to FIG. 30B, an augment construct 629 is shown according to an example aspect. As shown in FIG. 30B augment construct 629 includes only augment 630. However, in other embodiments, augment construct 629 may include additional components. Augment 630 may be substantially similar to augment 600, except that a third edge of augment 630 may be closed, with only one end providing an opening into a cavity. For example, as shown in FIG. 30B, augment 630 defines a cavity 631 configured to receive a balloon implant (e.g., implant 110, implant 210, etc.). Cavity 631 is accessible from an opening on only one end 637 of augment 630 between edges 635 and 636. A first edge 632, a second edge 633, and a third edge 634 may all be closed. As shown in FIG. 30B, edges 632, 633, 634 are continuously closed, but as discussed above with respect to edges 602, 604 of augment 600, any or all of edges 632, 633, 634 may be non- continuously closed, with openings providing access to cavity 631 (which may not be large enough for an inflated balloon to pass therethrough). In some examples, open end 637 may be completely open from edge 632 to edge 634, with edges 635, 636 not coupled at all along the length of one end 637. In other aspects, edges 635, 636 may be partially coupled, for example, adjacent the edges 632, 634 such that the opening does not extend fully from edge 632 to edge 634. FIG. 40 illustrates one of these aspects, for example. In other examples, edges 635, 636 may be coupled (e.g., adhered, stitched, etc.) together after a balloon has been positioned within cavity 631 to close the opening. In other examples, augment 630 may include a suture or filament coupled to openings or eyelets at edges 635, 636 and surrounding the opening in end 637 that may act as a drawstring that may be tensioned to close the opening in end 637 after a balloon has been inserted into cavity 631. As discussed above with respect to FIG. 30A, in some examples, a suture or filament may be stitched through the augment 630 (or structure coupled to the augment 630) as opposed to being coupled to pre-existing openings or eyelets. For example, a suture or filament may encircle the opening, passing through the material of the augment 630 several times along the edges 635, 636 of the opening before being tensioned to close the opening after a balloon has been inserted into cavity 631.

As discussed above, augment construct 629 may include additional components in addition to an augment. For example, the augment construct 629 may include a receiver structure which may be formed into a shape similar to that of the augment 630 shown in FIG. 30B such that the receiver structure similarly defines a portion or portions of cavity 631, and the augment (which may be, for example, a patch similar to augment 120′) may be coupled to the receiver structure (e.g., to the bottom of the receiver structure). As another example, the augment construct 629 may include an augment that forms one side of the shape shown in FIG. 30B (e.g., the top or bottom surface), and a receiver structure that forms the remainder of the shape, such that the augment and the receiver structure cooperate to define the cavity 631. Augment construct 629 may further include filaments (e.g., sutures) used to stitch edges 632-634.

Augment 630 may be used in various ways in a surgical procedure as discussed above with respect to the other augments described herein and may be made with similar materials. For example, augment 630 may be similar to augment 600, and cavity 631 may be similar to internal cavity 610 of augment 600. Thus, when augment 630 is secured to tissue of a patient and a balloon is disposed and secured within the internal cavity 631 of augment 630, the balloon may also be secured to the tissue via augment 630. After augment 630 and the balloon are attached to the tissue, the balloon may be inflated. The volume of the internal cavity 631 and/or the flexibility of the augment 630 may be sufficient to allow inflation of the balloon. Augment 630 may be secured to tissue using any of the methods for coupling augments to tissue described herein. For example, fixation elements (e.g., u-shaped bars, staples, pins, screws, etc.) may be pressed through edges 632, 633, 634 of augment 630 and into tissue. Alternatively, filaments or sutures may be passed through edges 632, 633, 634 and coupled to tissue or anchors previously coupled to the tissue. In other examples, edges 632, 633, 634 may include openings or eyelets configured to receive a suture or filament for securing augment 630 to tissue. In still other examples, a balloon in the cavity 631 may be secured directly to tissue such that augment 630 is secured to tissue via the balloon. Augment 630 may be inserted into an implantation site using an implant delivery instrument.

Certain aspects of the present disclosure that relate to kits contemplate kits that include dual implant prostheses. In some embodiments, a kit may include two or more dual implant prostheses. In some examples, two or more of the dual implant prostheses may be the same. In other examples, two or more of the dual implant prostheses may be different. Differences may be in the form of size, arrangement and materials, for example. Any of the contemplated kit embodiments may include one or more augments and/or one or more receiver assemblies. A kit may also include one or more implant delivery tools. In other embodiments, a kit may include two or more augments or two or more receiver assemblies.

Aspects of the present disclosure that relate to implantation of a prosthesis include further embodiments that involve the use of one or more of a dual implant, a receiver assembly, and an augment. While the following embodiments describe placement of a prosthesis in a shoulder joint, it should be appreciated that such description is exemplary and that the prosthesis may also be implanted in other areas of the body.

In one embodiment, one dual implant prosthesis from among prostheses 300A-I is delivered to a shoulder joint of a patient. For example, prosthesis 300A-I may be delivered so that when implantation is completed, a first implant 310A-I of prosthesis 300A-I is positioned in the glenohumeral joint and a second implant 320A-I of prosthesis 300A-I is positioned in a subacromial space. Placement of an inflatable implant within the intra-articular joint space is advantageous in that it provides cushioning and relief to the patient, with similar advantages for an inflatable implant in the subacromial space. For the sake of brevity, a collective reference to the prosthesis is used in this description to indicate that any one prosthesis from among those referenced may be used.

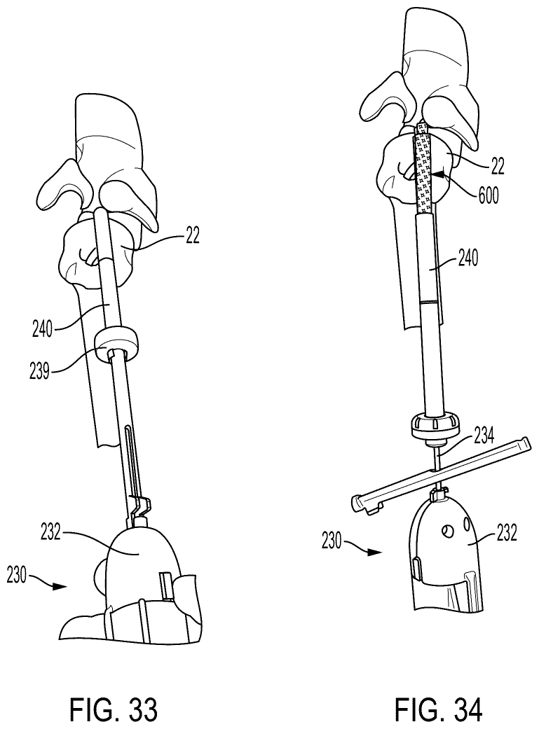

Turning to the steps of the method, initially, prosthesis 300A-I is loaded onto an implant delivery tool 230 outside of the patient. To do so, a sheath 240 of implant delivery tool 230, shown in FIG. 33, for example, is withdrawn toward handle 232, and prosthesis 300A-I is attached to an implant rod 234 exposed by the withdrawn sheath 240. Then, sheath 240 is slid back to its previous position, now covering prosthesis 300A-I. During the process of loading prosthesis 300A-I onto implant delivery tool 230, prosthesis 300A-I may be rolled up within sheath 240. Further, first and second implants 310A-I, 320A-I may be arranged within sheath 240 so that each is axially separated along a length of sheath 240. Implant delivery tool 230 is then directed through an access into the patient until it is positioned for delivery of the first of the two implants inside sheath 240. Sheath 240 is then retracted in vivo to release first implant 310A-I followed by second implant 320A-I. Optionally, implant delivery tool 230 may be used to adjust a position of one or both implants 310A-I, 320A-I after being released from the implant delivery tool 230 or implant delivery tool 230 may be removed and another instrument may be advanced into the implantation site area for such purpose. Prosthesis 300A-I may then be inflated. For prosthesis 300A, inflation may be via inflation port 312A to inflate both implants 310A, 320A. For prostheses 300B-300I, implant delivery tool 230 may be attached to respective inflation ports 312B-I, 322B-I to inflate both implants.

In one embodiment, each implant 310A-I, 320A-I of one prosthesis from among prostheses 300A-I is delivered into a patient separately and then combined in vivo. Thus, for example, first implant 310A-I may be loaded onto implant delivery tool 230 as described above then delivered to an implantation site. Once first implant 310A-I is in position within the patient, implant delivery tool 230 is withdrawn and loaded with second implant 320A-I, again, delivered to an implantation site near that for the first implant. At this juncture, a filament or other attachment structure may be delivered into the patient and used to attach first implant 310A-I to second implant 320A-I. Once the respective implants are attached to define a dual implant, each implant may be inflated.

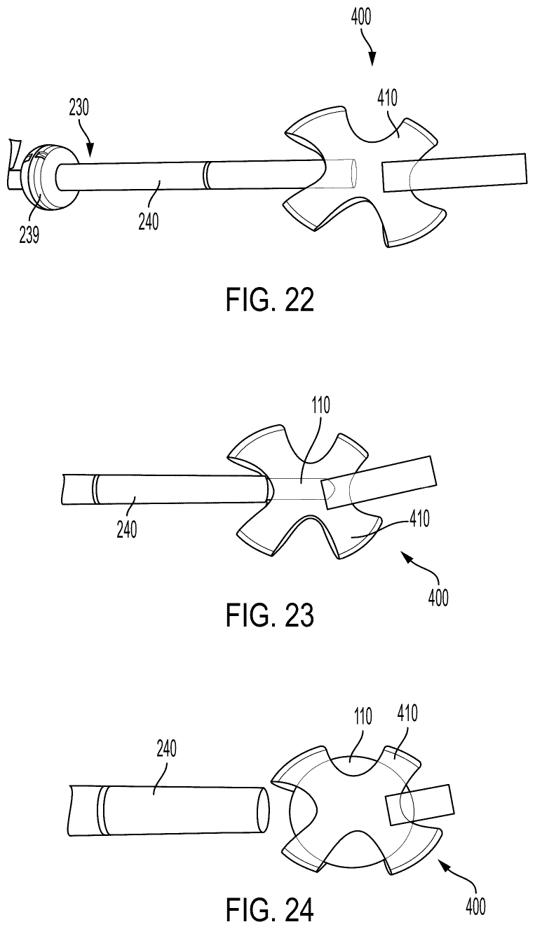

In other embodiments, a method of implanting a prosthesis may be performed with the use of a receiver assembly such as receiver assembly 400 or receiver assembly 500. In one embodiment, a single implant 110 may be delivered and implanted with receiver assembly 400. In this method, receiver assembly 400 may be loaded onto implant delivery tool 230 in the same manner as described above for prosthesis 300A-I, then delivered to an implantation site when exposed from sheath 240. Subsequently, with implant delivery tool 230 withdrawn from the patient, implant 110 may be loaded onto implant delivery tool 230 so that implant 110 may be delivered to receiver assembly 400 at the implantation site. Specifically, once sheath 240 loaded with implant 110 is in the patient and positioned adjacent to body 410 so that its tip is in between upper and lower parts of body 410, as shown in FIG. 22, sheath 240 may be withdrawn to expose implant 110 into an internal volume of body 410, as shown in FIG. 23. Implant 110 may then be inflated via implant delivery tool 230, pushing outward the inner surfaces of body 410. Then, implant delivery tool 230 is removed, as shown in FIG. 24. In a variation of this embodiment, implant 110 may be loaded into receiver assembly 400 and then into implant delivery tool 230 outside of the patient. Then, when implant delivery tool 230 is advanced to an implantation site, withdrawal of sheath 240 allows for the delivery of implant 110 already retained within receiver assembly 400.

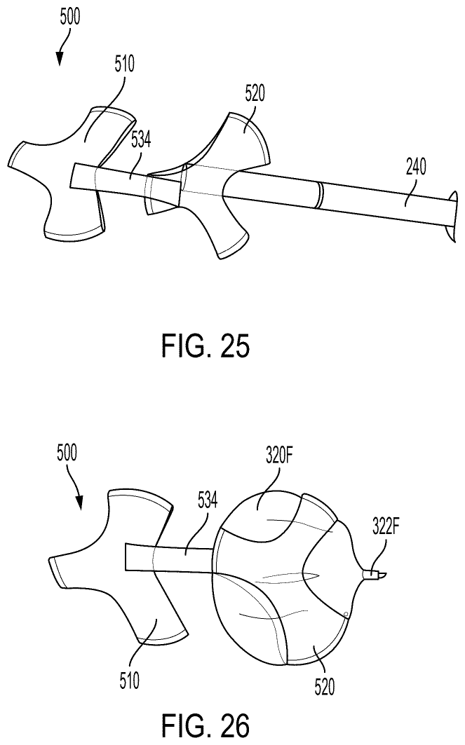

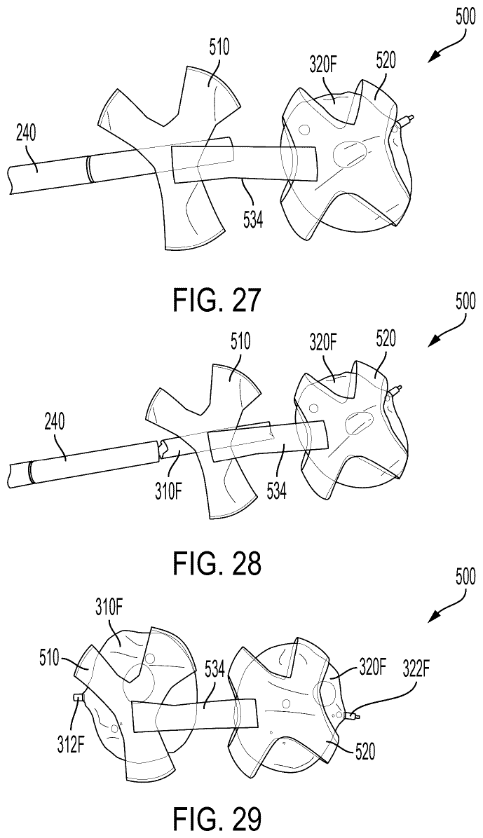

In some embodiments, a dual implant prosthesis, such as one prosthesis from among prostheses 300A-300I, may be implanted with receiver assembly 500. In one embodiment, a method of implanting prosthesis 300F is shown in part through FIGS. 25-29. Initially, receiver assembly 500 is loaded onto implant delivery tool 230 and then enclosed within sheath 240 to deliver and release receiver assembly 500 to an implantation site. Loading and delivery of receiver assembly 500 may be through the same process as described above for a dual implant. Implant delivery tool 230, after release of receiver assembly 500, is then withdrawn and loaded with second implant 320F outside of the patient. Second implant 320F is then delivered to an internal volume of second body 520 of receiver assembly 500, as shown in FIGS. 25-26. The aforementioned step may be performed in a variety of ways to customize a position of inflation port 322F in a specific cutout of second body 520. Such customization may be desirable to optimize a location of inflation port 322F for inflation of second implant 320F. Other considerations may include case of access for initial delivery of second implant 320F. This versatility in approach for delivering implants 310F, 320F renders it easier to deliver receiver assembly 500 in a separate step before delivery of implants 310F, 320F. The implant delivery tool 230 is once again withdrawn and then loaded with first implant 310F outside of the patient. First implant 310F is then delivered to an internal volume of first body 510 in the same manner as described for second implant 320F, as shown in FIGS. 27- 29. In some examples, first implant 310F may be delivered through a first access portal into the patient while second implant 320F is delivered through a second access portal into the patient. In other examples, first and second implants 310F, 320F may be delivered through the same access portal. In examples where a single access portal is used, a location of implant delivery tool during performance of the method may be different from that shown in FIGS. 25-29. Inflation of each implant 310F, 320F may be performed either after delivery as shown in FIG. 29 or at an earlier step for second implant 320F, as deemed appropriate for the surgery at issue. In a variation of the above described method, the method may be performed by initially delivering first implant 310F followed by second implant 320F. It should be appreciated that references to prosthesis 300F are exemplary and that this method may be performed with any contemplated dual implant prosthesis.

In another embodiment, a method of implanting prosthesis 300F or any one prosthesis from among prostheses 300A-I, with receiver assembly 500 may proceed as follows. A single receiver assembly, such as receiver assembly 400, may first receive a single implant, such as first implant 310F such that first implant 310F is disposed within body 410 of receiver assembly 400. Then, the combined structure is loaded onto an end of implant delivery tool 230 with sheath 240 withdrawn, and subsequently enclosed by sheath 240, each of the aforementioned steps taking place outside of the patient. Then, implant delivery tool 230 is used to deliver first implant 310F to a desired implantation location within a patient. Once first implant 310F is appropriately positioned, implant delivery tool 230 is detached and withdrawn from the patient. Then, a second implant 320F is received in another receiver assembly 400 and the process is repeated again, this time for second implant 320F. After each implant 310F, 320F is delivered and released, there are two receiver assemblies 400 in the patient, each enclosing a respective implant 310F, 320F. The method continues by joining the two receiver assemblies 400 to form a single receiver assembly 500. Such joinder may be accomplished with the use of a filament or another attachment structure, e.g. supplemental material segment 534 or through another form of attachment as described elsewhere in the present disclosure. Once receiver assemblies are attached, the user may proceed to inflate the respective first and second implants 310F, 320F. Optionally, such inflation may occur earlier in the method, although in many cases it may be desirable to inflate the implants late in the procedure to preserve as much operating space as possible.

In another embodiment, a method of implanting prosthesis 300F or any prosthesis from among prostheses 300A-I with receiver assembly 500 may proceed as follows. Outside of the patient, a first implant 310F may be received in a first body 510 of receiver assembly 500 and a second implant 320F may be received in a second body 520 of receiver assembly 500. The combined structure may then be loaded onto an engagement end of implant delivery tool 230 with sheath 240 withdrawn. Once loaded, the combined structure may be rolled in a manner that allows for sheath 240 to be advanced back over receiver assembly 500 and implants 310F, 320F. In this arrangement, one implant will be closer to the tip of implant delivery tool 230 than the other to allow for sequential delivery. Implant delivery tool 230 is then advanced into the patient to an implantation site for the implant at the leading end of implant delivery tool 230 and a first of the two implants is released, already enclosed within a respective body of the receiver assembly 500. Delivery continues with release of the remaining implant and body of the receiver assembly 500 from the implant delivery tool 230. Once any optional adjustment of a position of either implant 310F, 320F is made, first and second implants 310F, 320F are inflated, expanding the bodies 510, 520 of receiver assembly 500, and implant delivery tool 230 is removed. In a variation of this method, an implant delivery tool with a long shaft may be used to perform the method such that the receiver assembly and the prosthesis may remain separate but both simultaneously disposed within the sheath. In this manner, upon positioning of the sheath at the implantation site, the sheath may be partially retracted to deliver the receiver assembly, then, once a free end of the sheath is appropriately positioned into an internal volume of a body of receiver assembly, may be further retracted to deliver an implant of the prosthesis into such internal volume to be held by the body. This step may be repeated where there are two bodies on a receiver assembly to receive two implants.

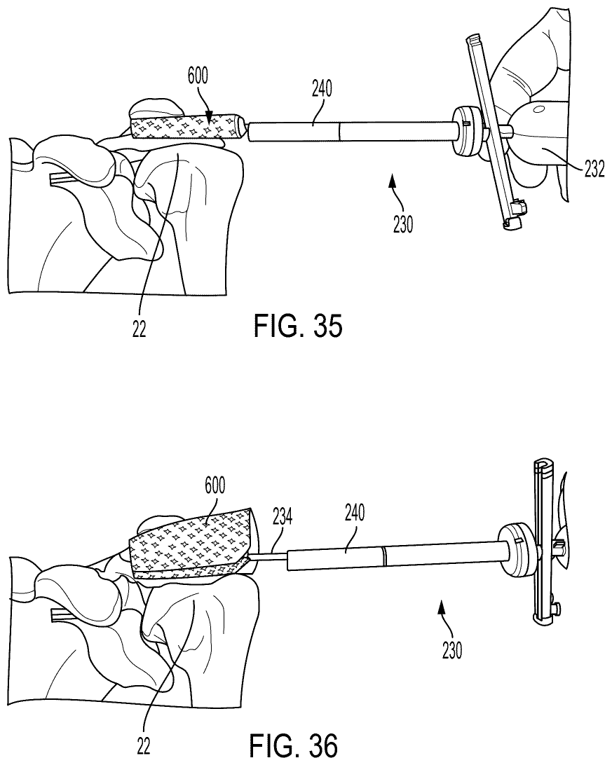

In still further method embodiments, an implant as contemplated by the present disclosure may be complemented by augment 600 in its implanted condition. In one embodiment, augment 600, as shown in FIG. 30A, may be loaded into sheath 240 of implant delivery tool 230 and delivered to an implantation site within a patient, i.e., a site for receipt of an implant. After augment 600 is released into position within the patient, implant delivery tool 230 is removed and then loaded with a dual implant prosthesis, such as one prosthesis from among prostheses 300A-I. The prosthesis 300A-I is then delivered into the patient to be received within cavity 610 of augment 600. Once prosthesis 300A-I is inside cavity 610, it may be inflated. In a variation of this embodiment shown in FIGS. 33-36, the dual implant prosthesis may be disposed in cavity 610 of augment 600 before both are loaded together into sheath 240 of implant delivery tool 230. In such an arrangement, augment 600 and prosthesis 300A-I are delivered together to the implantation site. Once in position at the implantation site, implant delivery tool 230 may be used to inflate prosthesis 300A-I within augment 600, as shown in FIG. 36. In a further variation of this embodiment that also includes delivery of prosthesis 300A-I and augment 600 at the same time, an implant delivery instrument may be utilized that includes a sheath of sufficient length so that the prosthesis 300A-I and augment 600 may be separately disposed along a length of the sheath. Namely, prosthesis 300A-I may be loaded first followed by augment 600, with augment 600 being positioned closer to a free end of sheath. In this manner, a partial retraction of sheath allows for the release of augment 600, while further retraction then allows for separate release of prosthesis 300A-I.

In other embodiments, methods of implant placement may include other instrumentation arrangements. In some of these methods, an implant is placed with augment 220. In one example, a prosthesis, e.g., one of prostheses 300A-3001, may be loaded into sheath 240 of implant delivery tool 230. Additionally, implant delivery tool 230 may further include augment sleeve 252, positioned over sheath 240 and attached to the same retractable base structure 239 on implant delivery tool 230. Further, augment 220, with filaments 226 attached thereto, may be positioned so that filaments 226 wrap around an outer surface of augment sleeve 252, with augment 220 held by an augment delivery tool 250 positioned adjacent to implant delivery tool 230. This arrangement may be the same as that shown in FIG. 6, but with prosthesis 300A-I in place of implant 210. The combined instrumentation is then delivered to the implantation site. In a variation, augment sleeve 252 may be a separate structure from implant delivery tool 230, augment sleeve 252 being inserted into the patient together with augment delivery tool 250. When the method is performed this way, an initial step includes wrapping filaments 226 around augment sleeve 252 and holding augment 220 with augment delivery tool 250. In this variation, once augment 220 is positioned at the implantation site, implant delivery tool 230 may be separately inserted so that sheath 240 is advanced into augment sleeve 252 in vivo. Once at the implantation site, the sheath 240 of implant delivery tool 230 and augment sleeve 252 are withdrawn together, causing prosthesis 300A-I to be exposed from sheath 240 and filaments 226 to settle over prosthesis 300A-I. In this manner, prosthesis 300A-I is within the strap enclosure formed by filaments 226. Further, the presence of augment delivery tool 250 maintains alignment of augment 220 to prosthesis 300A-I. Prosthesis 300A-I is then inflated, and even after inflation, remains retained by filaments 226 to maintain its position at the implantation site. In variations of this example, prosthesis 300A-I loaded into sheath 240 may first be received within a receiver assembly 500 such that when prosthesis 300A-I is delivered, it is already held within an internal volume of receiver assembly 500, in turn, within filaments 226 of augment 220. Although not shown in the figures, the augment delivery tool 250 may include a release mechanism to release attached objects, such as an augment. Further, it should be appreciated that when used in conjunction with each other, implant delivery tool 230 and augment delivery tool 250 may be referred to as an implant delivery system.

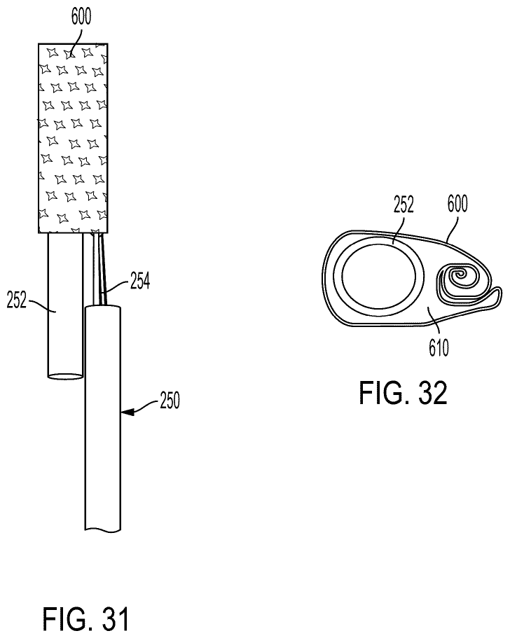

In some embodiments, a method using an implant delivery tool 230, an augment sleeve 252, and an augment delivery tool 250 may be performed to deliver an implant and augment 600. The method may proceed with loading a prosthesis, e.g., one of prostheses 300A-300I, onto implant delivery tool 230 so that prosthesis 300A-I may be enclosed by sheath 240. In this embodiment, implant delivery tool 230 also includes augment sleeve 252 attached over sheath 240, and the method proceeds with augment 600 being positioned over augment sleeve 252 and an augment rod 254 of augment delivery tool 250, as shown in part in FIG. 31 and in section in FIG. 32. At this loading stage, sheath 240 and augment sleeve 252 separate prosthesis 300A-I from augment 600. To position augment 600 in this way, augment rod 254 is kept close to augment sleeve 252. Then, implant delivery tool 230 and augment delivery tool 250 are directed into the patient and to an implantation site. At the implantation site, sheath 240 and augment sleeve 252 are withdrawn together to cause prosthesis 300A-I to be left within cavity 610 of augment 600. Then, prosthesis 300A-I may be inflated and any remaining instrumentation removed from the patient. In a variation similar to that described above, these embodiments may also be performed by first inserting augment sleeve 252 with augment delivery tool 250 to deliver augment 600, followed by separate insertion of implant delivery tool 230 such that sheath 240 is advanced into a lumen of augment sleeve 252 in vivo. This is followed by withdrawal of the sheath to expose prosthesis 300A-I within cavity 610 of augment 600, the remaining steps being the same. In yet another variation, a method may include delivering augment 600 solely with the use of augment delivery tool 250, then using augment delivery tool 250 to open up cavity 610 of augment 600 in vivo, such opening being prepared so that implant delivery tool 230 may be directed to cavity 610 to deliver prosthesis 300A-I into cavity 610.

In some embodiments, a method of implanting a prosthesis with augment 600 may include the use of an implant delivery tool that includes a central rod, a sheath slidable between a retracted and extended position along the rod, and an outer tube disposed over the sheath and independently slidable between a retracted position and an extended position along the rod. Outside of the patient, a prosthesis, e.g., one of prostheses 300A-300I, is loaded onto the rod and enclosed by the sheath. With outer tube still retracted, augment 600 is loaded onto an exterior of sheath, and then outer tube is extended to enclose sheath. The implant delivery tool is then advanced to the implantation site, where the outer tube is initially retracted to release augment 600, then the sheath is separately retracted to expose prosthesis 300A-I within cavity 610 of augment 600. The method may proceed from here with inflation of prosthesis 300A-I and removal of instrumentation from within the patient. While the examples discussed above each refer to the insertion of a prosthesis 300A-300I into the cavity 610 of 600, it should be understood that similar methods may be performed to insert a prosthesis 300A-300I into the cavity 631 of augment 630.

The methods of implantation of a prosthesis, such as a dual implant prosthesis, may be varied in many ways. For example, any one of the contemplated methods may further include an augment attached to a prosthesis and or a receiver assembly, where the augment is configured to stimulate soft tissue regrowth and healing. Such augment may be attached through any means as described throughout the present disclosure. For example, a suture may be used to secure an augment to a receiver assembly. Further, in any one of the contemplated method embodiments, the implant delivery tool used may be implant delivery tool 130 shown in FIG. 3 in place of implant delivery tool 230. In some examples, the method of implantation may be supplemented with fixation of a receiver assembly or augment to tissue. Such fixation may be temporary or intended to remain post-operatively over a longer duration. A filament, a staple, or other biocompatible fixation means may be used for such purpose. For example, a peripheral region of a receiver assembly may receive a filament that is also attached to tissue to hold the receiver assembly in a desired position relative to tissue. It should be appreciated, however, that although fixation of various implanted components is contemplated, the dual implant prosthesis itself provides stability and is expected to remain in its implanted position without fixation.

In some examples, an augment construct may include an augment coupled to or integrally formed with a second balloon in addition to the balloon of the balloon implant and may be referred to as a balloon augment. For example, the augment itself may be a balloon, or an augment may be mechanically or chemically attached to a separate balloon structure (e.g., similar to the attachment of the augment 120′ to the substrate 120). The balloon of the balloon augment may be inflated in order to improve the unfolding or unrolling of the balloon augment after being delivered to the implantation site in a collapsed (e.g., rolled, folded, etc.) configuration. The balloon of the balloon augment may be deflated after unrolling. In some examples, the balloon augment and/or the balloon implant may be delivered in collapsed (e.g., rolled, folded, etc.) configurations using an implant delivery instrument or tool. In some examples, the balloon augment or balloon implant may be positioned within a sheath of the instrument while in a collapsed, deflated configuration. The augment and/or the balloon may be released from the sheath and allowed to expand to an expanded configuration once positioned at the implantation site. In other examples, the balloon augment and/or the balloon implant may be inserted into an implantation site using an instrument that does not include a sheath, for example by wrapping the balloon augment or balloon implant around a shaft or holding the balloon augment or balloon implant with forceps or a similar instrument without the need for a sheath. In other examples, flexible members, sheets, and/or membranes or movable arms may be used to grip or hold the augment construct in the collapsed configuration by clamping, pinching, suturing, and/or piercing the augment construct. In still other examples, the balloon augment and/or the balloon implant may be inserted by hand without the use of an instrument. In some examples, filaments may encircle and hold the balloon augment and/or the balloon implant in their respective collapsed configurations, and the filaments may be cut when the balloon augment and/or the balloon implant have been delivered to the implantation site.

In some examples, the balloon of the balloon augment may be inflated with a therapeutic fluid to promote healing of the tissue at the implantation site. For example, the therapeutic fluid may include Hyaluronic acid, platelet-rich plasma, and/or bone marrow aspirate concentrate. The balloon of the balloon augment may be punctured after being positioned at the implantation site to release the therapeutic fluid. For example, the balloon may be punctured by a fixation member (e.g., a staple, a screw, a pin, a suture, etc.) in the process of coupling the balloon augment to the tissue at the implantation site.

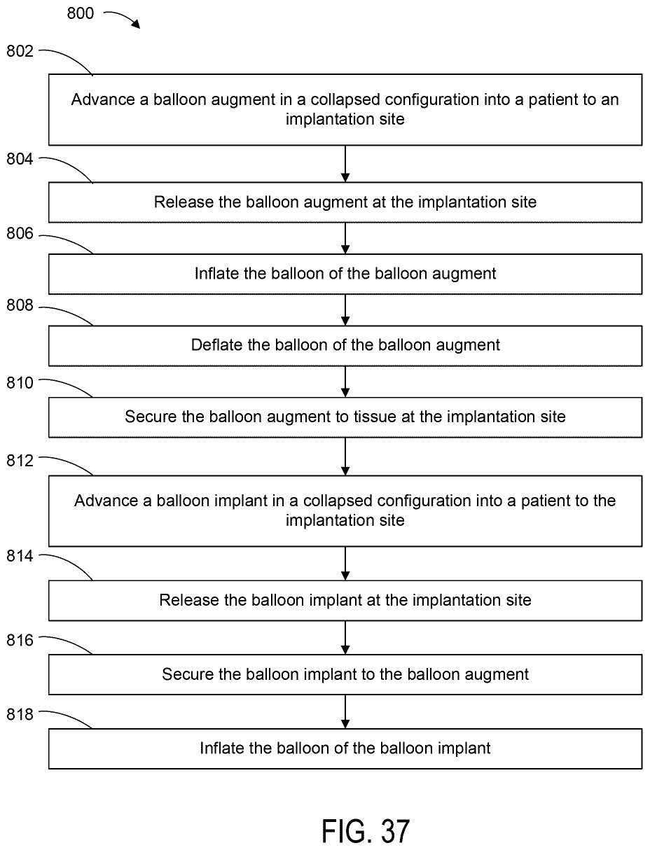

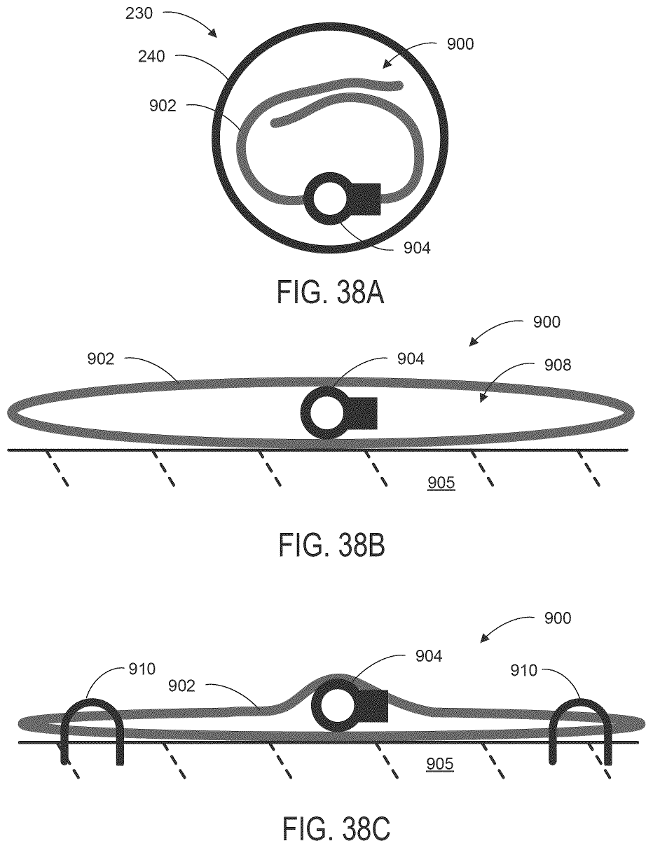

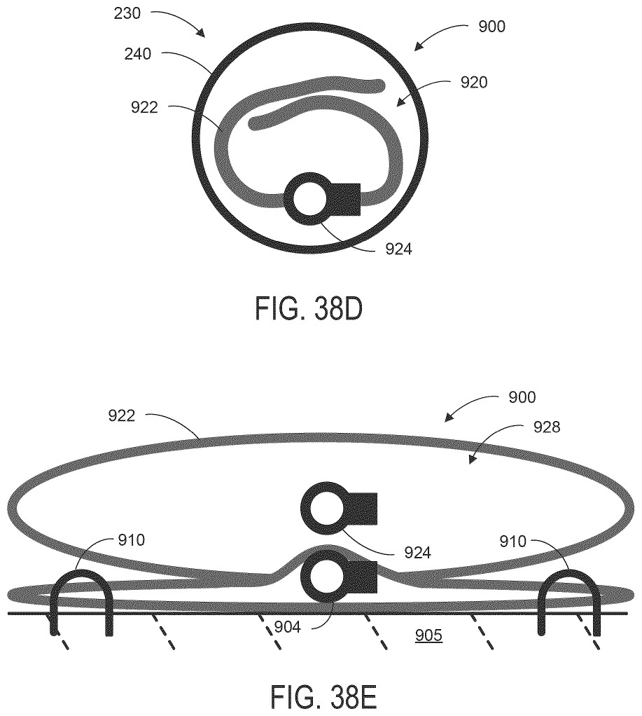

Referring now to FIG. 37, a flowchart illustrating an exemplary method 800 of delivering a balloon implant according to some embodiments is shown. An example of the method 800 is shown in FIGS. 38A-38E, in which at least one implant delivery tool 230 is used to deliver a balloon augment 900 and a balloon implant 920 to an implantation site in a patient. While the example shown in FIGS. 38A-38E illustrates one embodiment of the method 800, it should be understood that in alternative embodiments the balloon augment and the balloon implant may be advanced into the patient in their respective collapsed configurations using a different implant delivery instrument or without using an implant delivery instrument at all, as discussed above.

At operation 802 of the method 800, a balloon augment is advanced in a collapsed configuration into a patient to an implantation site. As discussed above, the balloon augment may be an augment construct including an augment formed into a balloon or an augment coupled to a sperate balloon structure. In the collapsed configuration, a balloon of the balloon augment may be in a deflated condition, and the balloon augment may be rolled or folded to allow the balloon augment to be inserted through a portal into a patient. For example, as shown in FIG. 38A, a balloon augment 900 is rolled within the sheath 240 of the implant delivery tool 230, which may then be advanced to an implantation site of a patient. In other examples, a balloon augment may be advanced into a patient using an implant delivery instrument without inserting the balloon augment into a sheath. For example, the balloon augment may be wrapped around a shaft that can be inserted into the patient or inserted into the patient using forceps or a similar instrument without the need for a sheath. In other examples, flexible members, sheets, and/or membranes or movable arms may be used to grip or hold the augment construct in the collapsed configuration by clamping, pinching, suturing, and/or piercing the augment construct. In still other examples, the balloon augment may be advanced into the patient by hand, without the use of an implant delivery instrument. In some embodiments, filaments may encircle and hold the balloon augment in the collapsed configuration, and the filaments may be cut in a subsequent operation (e.g., operation 804) to release the balloon augment.

In the example shown in FIG. 38A, balloon augment 900 includes a balloon 902 (e.g., a first balloon) and a valve assembly 904 fluidly coupled to and configured to allow fluid to be delivered into an inner cavity 908 of the balloon 902. For example, fluid may be supplied to the valve assembly 904 via the implant rod 234 of the implant delivery tool 230 or via a conduit of a different implant delivery instrument. In some examples, the balloon augment may include a balloon with an augment as described above (e.g., augment 120) coupled thereto. In other examples, the balloon itself may be an augment made of the materials discussed above with respect to other augments (e.g., collagen, cross-linked collagen, non-cross-linked collagen, reconstituted collagen, native collagen, human collagen, bovine collagen, xenograph collagen, a synthetic material, a polyester material, an absorbable material, an organic material, silk, or combinations thereof).

At operation 804 of the method 800, the balloon augment is released at the implantation site. Releasing the balloon augment may allow the balloon augment to expand at least partially from the collapsed configuration, for example, by at least partially unrolling or unfolding. For example, the sheath 240 of the implant delivery tool 230 may be retracted to release the balloon augment 900. The balloon augment 900 may remain coupled to the implant delivery tool 230 via the connection of the valve assembly 904 to the implant rod 234. In other embodiments, as discussed above, filaments encircling the balloon augment in the collapsed configuration may be cut to release the balloon augment. In other embodiments, releasing the balloon augment may include releasing the balloon augment from the grip of forceps or fingers.

At operation 806 of the method 800, the first balloon is at least partially inflated. In some examples, the first balloon may be inflated with a gas or fluid, such as air, carbon dioxide, saline solution, or other types of gases or fluids. In some examples, as discussed above, the first balloon may be inflated with a therapeutic fluid such as Hyaluronic acid, platelet-rich plasma, bone marrow aspirate concentrate, and/or other therapeutic fluids. Inflation of the first balloon may cause the balloon augment to fully unroll or unfold, whereas, in some examples, an augment may not fully unfold or unroll after being released (e.g., from the implant delivery instrument) without further manipulation of the augment. FIG. 38B shows the balloon augment 900 after the balloon 902 has been inflated at an implantation site above tissue 905 by, for example, supplying gas or fluid to the inner cavity 908 via the valve assembly 904. In some examples, the implant delivery instrument may provide the gas or fluid to an inner cavity of the balloon. For example, the implant rod 234 may include a conduit configured to provide the gas or fluid to the balloon 902 via the valve assembly 904. If a similar implant delivery instrument is not used to deliver the balloon augment 900, a separate inflation conduit may be coupled to the valve assembly 904 and used to deliver fluid to inflate the balloon 902.

At operation 808 of the method 800, the first balloon is at least partially deflated, and at operation 810, the balloon augment is secured to tissue at the implantation site. In some examples, deflating the first balloon in operation 808 may include withdrawing the fluid or gas from the inner cavity of the first balloon via a valve (e.g., valve assembly 904). In other examples, deflating the first balloon in operation 808 may include puncturing the balloon, which may release the fluid used to inflate the balloon (e.g., therapeutic fluid) from the inner cavity of the balloon. In some examples, operations 808 and 810 may be performed substantially simultaneously by pressing a fixation element (e.g., a staple, a screw, a pin, a suture etc.) through the balloon augment and into the tissue at the implantation site, puncturing the balloon in the process. For example, as shown in FIG. 38C, two staples 910 are pressed through the balloon augment 900, puncturing the balloon 902, which may release therapeutic fluid from the inner cavity 908 (i.e., through the holes formed in the balloon 902 by the two staples 910). In some embodiments, method 800 may not include operation 808, and balloon of balloon augment 900 may not be deflated. For example, balloon augment 900 may remain inflated or partially inflated (e.g., if only partially inflated at operation 806) throughout method 800 and after the implantation of the balloon augment and balloon implant is completed.

At operation 812 of the method 800, a balloon implant is advanced in a collapsed configuration into a patient to an implantation site. In the collapsed configuration, a balloon of the balloon implant may be in a deflated condition, and the balloon implant may be rolled or folded to allow the balloon implant to be inserted through a portal into a patient. In some examples, the same delivery instrument may be used to deliver the balloon augment and the balloon implant in sequence. In other examples, a different delivery tool may be used to deliver the balloon implant than the delivery tool used to deliver the balloon augment. For example, the augment delivery tool 250 may be used to deliver the balloon augment, and the implant delivery tool 230 may be used to deliver the balloon implant. In other examples, the balloon augment and/or the balloon implant may be advanced into the patient by hand, without the use of an implant delivery instrument. In the example shown in FIG. 38D, a balloon implant 920 is rolled within the sheath 240 of the implant delivery tool 230, which may then be advanced to the implantation site of the patient. The balloon implant 920 includes a balloon 922 (e.g., a second balloon) and a valve assembly 924 fluidly coupled to and configured to allow fluid to be delivered into an inner cavity 928 of the balloon 922. In some examples, fluid may be supplied to the valve assembly 924 via the implant rod 234 of the implant delivery tool 230. The balloon implant 920 may be similar or equivalent to the balloon implants/prostheses discussed above (e.g., prosthesis 100).

At operation 814 of the method 800, the balloon implant is released at the implantation site. As discussed above with respect to the balloon augment, releasing the balloon implant may allow the balloon implant to expand at least partially from the collapsed configuration, for example, by at least partially unrolling or unfolding. For example, the sheath 240 of the implant delivery tool 230 may be retracted to release the balloon implant 920. The balloon implant 920 may remain coupled to the implant delivery tool 230 via the connection of the valve assembly 924 to the implant rod 234. In other embodiments, as discussed above, filaments encircling the balloon in the collapsed configuration may be cut to release the balloon release. In other embodiments, releasing the balloon may include releasing the balloon augment from the grip of forceps or fingers.

At operation 816 of the method 800 the balloon implant is secured to the balloon augment. For example, balloon implant 920 may be secured to balloon augment 900, e.g., using various techniques described herein. For example, the balloon implant 920 may be chemically coupled to the balloon augment 900 using an adhesive such as a fibrin glue, a cyanoacrylate, or combinations thereof. In other examples, the balloon implant 920 may be mechanically coupled to the balloon augment 900, for example, using filaments (e.g., sutures) as discussed above. For example, the balloon implant 920 may include tabs that can be secured to the deflated balloon augment 900 using filaments (e.g., sutures). In some examples, the balloon implant 920 may be coupled directly to the tissue 905 instead of or in addition to being coupled to the balloon augment 900. For example, the balloon implant 920 may be positioned on top of the balloon augment 900 but not coupled thereto, and filaments coupling the balloon implant 920 to tissue 905 may restrain the movement of the balloon implant 920 such that the balloon implant 920 remains positioned on top of balloon implant 920.

At operation 818 of the method 800, the second balloon (i.e., the balloon of the balloon implant) is inflated. In some examples, the second balloon may be inflated with a gas or fluid, such as air, carbon dioxide, saline solution, or other types of gases or fluids. In some examples, as discussed above, the first balloon may be inflated with a therapeutic fluid such as Hyaluronic acid, platelet-rich plasma, bone marrow aspirate concentrate, and/or other therapeutic fluids. As shown in FIG. 38E, the balloon implant 920 is shown after the second balloon 922 has been inflated at the implantation site above the balloon augment 900 by, for example, supplying gas or fluid to the inner cavity 928 via the valve assembly 924. In some examples, the second balloon may be inflated before the balloon implant is coupled to the balloon augment (e.g., operation 818 may be performed before operation 816). Inflation of the second balloon may cause the balloon implant to fully unroll or unfold, whereas, in some examples, an implant may not fully unfold or unroll after being released (e.g., from the implant delivery instrument) without further manipulation of the implant. As discussed above with respect to operation 806, the implant delivery instrument used to deliver the balloon implant may provide the gas or fluid to an inner cavity of the second balloon. For example, the implant rod 234 may include a conduit configured to provide the gas or fluid to the balloon 922 via the valve assembly 924. If a similar implant delivery instrument is not used to deliver the balloon implant 920, a separate inflation conduit may be coupled to the valve assembly 924 and used to deliver fluid to inflate the balloon 922.

As discussed above, while FIGS. 38A-38E illustrate an embodiment of a method in which the balloon augment and balloon implant are delivered in the sheath of an implant delivery instrument, in some examples, as discussed hereinabove, a different implant delivery instrument may be used with or without the use of a sheath, or no implant delivery instrument may be used to deliver the balloon augment and/or the balloon implant. For example, a balloon augment and/or a balloon implant may be rolled or folded and advanced to the implantation site by hand or with simple forceps or the like, without the use of an implant delivery instrument. Once positioned at the implantation site, the first balloon and/or the second balloon may be inflated by, for example, fluidly coupling a tube or syringe to the respective balloon (e.g., via the respective valve assembly 904, 924) and pumping fluid into the balloon. In other examples and as discussed above, an implant delivery instrument without a sheath may be used to deliver the balloon augment and/or the balloon implant.

In some examples, the first balloon (e.g., balloon 902) and/or the second balloon (e.g., balloon 922) may be partially inflated or may be partially deflated after being fully (or more fully) inflated. For example, the first balloon or the second balloon may first be fully inflated to promote unfolding or unrolling of the augment or implant, and then partially deflated by removing some of the inflation fluid to achieve a desired inflation pressure (e.g., to promote healing or pain relief in the joint. In some examples, the balloon augment may be secured to tissue at the implantation site (e.g., without puncturing or otherwise causing a loss of integrity of the first balloon), and the first balloon may remain in a partially (or fully) inflated state, rather than being deflated. Either or both of the first balloon or the second balloon may be fully or partially inflated and fully or partially deflated one or more times during the method 800 in order to manipulate the positions, orientations, and inflation pressures of the balloon augment and the balloon implant.

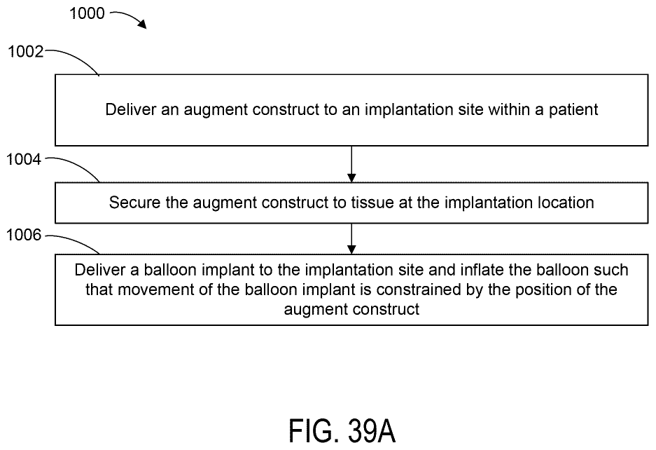

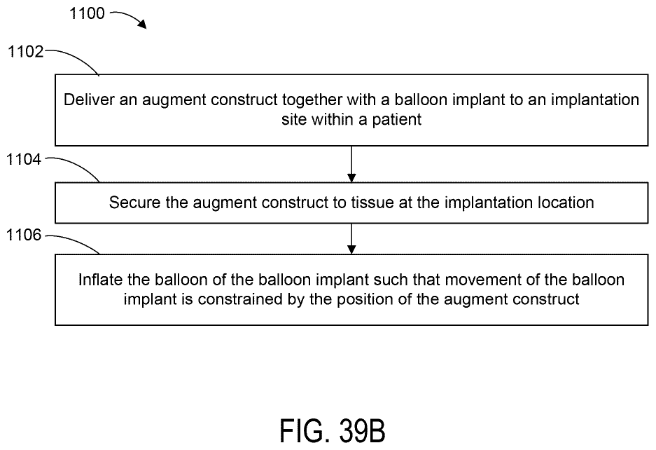

Referring now to FIG. 39A, a flowchart illustrating an exemplary method 1000 of delivering a balloon implant according to some embodiments is shown. In the method 1000, an augment construct is delivered to the implantation site prior to delivery of the balloon implant. As discussed above, the augment construct may include only an augment or may include an augment as well as one or more additional structures. The balloon implant is then delivered and positioned relative to the augment construct such that, when the balloon of the balloon implant is inflated, movement of the balloon implant is restrained based on a position of the augment construct. For example, the balloon implant may be secured to the augment construct using filaments or other fixation members (e.g., staples, pins, screws, etc.). In other examples, the balloon implant may be positioned within a receptacle of the augment construct. For example, the augment construct may include an inner cavity defining a receptacle configured to receive and retain the balloon implant, or the augment construct may include filaments coupled thereto, and the receptacle may be defined between the filaments and the augment. The various operations of the method 1000 are discussed above with respect to augments 120, 220, 600, 630 and the body 410 of receiver assembly 400 or the bodies 510, 520 of receiver assembly 500 (each of which may be an augment or may be coupled to an augment) for purposes of illustration.

At operation 1002 of the method 1000, an augment construct is delivered to an implantation site within a patient. For example, as discussed above, an augment construct including an augment (e.g., augments 120, 220, 600, 630 and the body 410 of receiver assembly 400 or the bodies 510, 520) may be delivered to the implantation site using an augment delivery tool 250 or using the implant delivery tool 230. For example, the augment construct may be delivered within a sheath of a delivery instrument or may be wrapped around a shaft of a delivery instrument without the need for a sheath. In other examples, the augment may be delivered by hand or with the use of various surgical instruments rather than a dedicated delivery instrument, as discussed above with respect to method 800. For example, flexible members, sheets, and/or membranes or movable arms may be used to grip or hold the augment construct in the collapsed configuration by clamping, pinching, suturing, and/or piercing the augment construct, as discussed hereinabove.

At operation 1004 of the method 1000, the augment construct is secured to tissue at the implantation site. For example, as discussed above, an augment construct may be mechanically secured to tissue at an implantation site using fixation elements (e.g., u-shaped bars, staples, pins, screws, etc.) or using filaments or sutures to couple the augment construct to tissue or to anchors previously coupled to the tissue. The fixation elements may be pressed (or screwed, etc.) through material of the augment construct or passed through a predefined opening in the augment construct and pressed (or screwed, etc.) into tissue at the implantation site. Filaments or sutures may similarly be passed through predefined openings in the augment construct or directly through the material of the augment construct and used to secure the augment construct to the anchors (e.g., by tying knots in the filaments or using knotless fixation anchors or techniques such as splices or finger traps).

At operation 1006, a balloon implant is delivered to the implantation site and the balloon of the balloon implant is inflated such that the augment construct positionally restrains movement of the balloon implant. For example, when augment construct 599 has been coupled to tissue at the implantation site, delivering the balloon implant (e.g., implant 110, implant 210, etc.) includes inserting the balloon implant into the cavity 610. Similarly, when the augment construct 629 has been coupled to tissue at the implantation site, delivering the balloon implant includes inserting the balloon implant into the cavity 631. Upon inflating the balloon of the balloon implant, the inner walls of the respective cavity 610, 631 may restrain movement of the balloon implant, for example, such that the balloon implant is captive within the respective cavity 610, 631. In some examples, the balloon implant may be inflated to a size that is larger than the size of the openings to the respective cavity 610, 631. For example, according to some aspects, a dimension of the balloon implant, when inflated, may be larger than an adjacent dimension of the opening in order to positionally restrain the movement of the balloon implant. Thus, the opening into cavity 610 may be large enough to receive the balloon implant in a deflated, collapsed configuration but not so large as to allow the implant to be removed from the cavity 631 when in an inflated, expanded configuration (e.g., without tearing the augment construct 599 or using substantial force beyond what is expected during normal use of the joint in which the implant is implanted). For example, in some embodiments, similar to other examples above, there may be at least one point within the inflated implant wherein every cross-section of the implant passing through that point has a larger area than the area of the opening, such that the inflated implant cannot fit through the opening in any orientation. In some embodiments, the inflated implant is larger in two perpendicular dimensions passing through at least one point within the inflated implant than a maximum dimension of the opening, such that the inflated implant cannot fit through the opening in any orientation. In some embodiments, a portion of the implant adjacent to the opening is larger than (e.g., has a larger cross-sectional area than) the opening.

In some examples, delivering the balloon implant may include closing at least one of the openings of the augment construct 599, 629 (e.g., by stitching the opening at least partially shut, at least partially closing the opening by tensioning suture or filament like a drawstring, etc.) which may further retain the balloon implant within the respective cavity 610, 631 by reducing the size of the opening. In these embodiments, delivering the balloon implant to the respective cavity 610, 631 and/or inflating the balloon causes the augment construct to positionally restrain movement of the balloon implant because, as discussed above, the inflated balloon may be larger than the openings to the cavity 610, 631.

As another example of operation 1006, when augment construct 219 (as shown in FIG. 10) has been coupled to tissue at the implantation site, the balloon implant (e.g., implant 110, implant 210) may be inserted between the augment 220 and the filaments 226. Positioning the balloon implant between the augment 220 and the filaments 226 and/or inflating the balloon secures the balloon implant to the augment construct 219 and causes the augment construct 219 to positionally restrain the movement of the balloon implant to be restrained relative to the augment 220. Thus, the balloon implant is restrained by the plurality of filaments 226 or the plurality of filaments 226 in combination with the augment 220 after the balloon implant has been inflated. For example, the balloon implant may be at least partially restrained when positioned between the augment 220 and the filaments 226 in a deflated, expanded (e.g., unfolded, unrolled, etc.) configuration. Inflating the balloon of the balloon implant may further secure the balloon implant to the augment 220 as the volume of the balloon implant increases and the filaments 226 tension around the balloon implant. For example, according to some aspects, a dimension of the balloon implant, when inflated, may be larger than an adjacent dimension of the spaces between the filaments 226 in order to positionally restrain the movement of the balloon implant 110, In some embodiments, similar to other examples above, there may be at least one point within the inflated implant wherein every cross-section of the implant passing through that point has a larger area than the area of the spaces between the filaments 226, such that the inflated implant cannot fit through the spaces in any orientation. In some embodiments, the inflated implant is larger in two perpendicular dimensions passing through at least one point within the inflated implant than a maximum dimension any of the spaces between the filaments 226, such that the inflated implant cannot fit through the spaces in any orientation. In some embodiments, a portion of the inflated implant 110 adjacent to a space between the filaments 226 is larger than (e.g., has a larger cross-sectional area than) the space between the filaments 226.

As another example of operation 1006, when the augment coupled to tissue at the implantation site is or is coupled to the body 410 of receiver assembly 400 (or one of the bodies 510, 520 of receiver assembly 500, as shown in FIGS. 20-29), delivering the balloon implant (e.g., implant 110, implant 320F) may include inserting the balloon implant into the cavity through one of the cutouts in the body 410. Positioning the balloon implant in the cavity and allowing the balloon implant to unroll or unfold from a collapsed configuration to an expanded configuration and/or inflating the balloon secures the balloon implant to the augment and causes the movement of the balloon implant to be restrained relative to the augment. For example, by inflating the balloon, the size (e.g., volume) of the balloon implant may increase such that the balloon implant can no longer fit through the cutout and cannot be easily removed from the internal cavity (e.g., without using substantial force or tearing the body 410). For example, in some embodiments, similar to other examples above, there may be at least one point within the inflated implant wherein every cross-section of the implant passing through that point has a larger area than the area of the cutouts, such that the inflated implant cannot fit through the cutouts in any orientation. In some embodiments, the inflated implant is larger in two perpendicular dimensions passing through at least one point within the inflated implant than a maximum dimension of the cutout. In some examples, a portion of the implant 110 adjacent to a cutout is larger than (e.g., has a larger cross-sectional area than) the cutouts.

In each of the above examples, fixation elements (e.g., staples, pins, screws, etc.), sutures, or filaments may also be used to mechanically couple the balloon implant to the augment or to tissue at the implantation site (in addition to the features of the augment that retain the balloon implant). Adhesive may also be used to couple the balloon implant to the augment at the implantation site. For example, adhesive may be applied to the inner cavity of an augment or to the balloon implant before the balloon implant is placed inside of the inner cavity.

As another example of operation 1006, when the augment 120 is coupled to tissue at the implantation site, delivering the balloon implant (e.g., implant 110, implant 210, etc.) may include mechanically coupling the balloon implant to the augment 120, for example, using sutures or filaments or other fixation elements (e.g., staples, pins, screws, etc.). In other examples, delivering the balloon implant (e.g., implant 110, implant 210, etc.) may include chemically (e.g., adhesively) securing the balloon implant to the augment 120. Securing the balloon implant to the augment causes the movement of the balloon implant to be restrained relative to the augment. The balloon may be inflated before or after the balloon implant is coupled to the augment 120.

Referring now to FIG. 39B, a flowchart illustrating an exemplary method 1100 of delivering a balloon implant according to some embodiments is shown. Method 1100 may be substantially similar to method 1000, except that the balloon implant and the augment construct may be delivered together, rather than first delivering the augment construct and then delivering the balloon implant. As discussed above, the augment construct may include only an augment or may include an augment as well as one or more additional structures.

At operation 1102 of the method 1100, an augment construct is delivered together with a balloon implant to an implantation site within a patient. The balloon implant may be positioned in a manner such that, when the balloon of the balloon implant is inflated it will be positionally restrained relative to the augment construct. In some examples, the balloon implant may be secured to the augment construct using filaments or other fixation members (e.g., staples, pins, screws, etc.). In other examples, the balloon implant may be positioned within a receptacle of the augment construct. For example, as discussed hereinabove, the augment construct may include an inner cavity defining a receptacle configured to receive the balloon implant, or the augment construct may include filaments coupled thereto, and the receptacle may be defined between the filaments and the augment.

At operation 1104 of the method 1100, the augment construct is secured to tissue at the implantation site. Operation 1104 may be substantially similar to operation 1004 except that the balloon implant is already coupled to or positioned within or proximate to the augment construct when the augment construct is secured to the tissue. As discussed above, an augment construct may be mechanically secured to tissue at an implantation site using fixation elements (e.g., u-shaped bars, staples, pins, screws, etc.) or using filaments or sutures to couple the augment construct to tissue or to anchors previously coupled to the tissue. The fixation elements may be pressed (or screwed, etc.) through material of the augment construct or passed through a predefined opening in the augment construct and pressed (or screwed, etc.) into tissue at the implantation site. Filaments or sutures may similarly be passed through predefined openings in the augment construct or directly through the material of the augment construct and used to secure the augment construct to the anchors (e.g., by tying knots in the filaments or using knotless fixation anchors or techniques such as splices or finger traps). At operation 1106 of the method 1100, the balloon of the balloon implant is inflated such that the augment construct positionally restrains movement of the balloon implant as discussed above with respect to operation 1006 of method 1100.

In one example of method 1100, implant 110 may be coupled to augment construct 120 in a deflated condition before augment construct 120 and implant 110 are inserted into the patient at operation 1102. Augment construct 120 may then be secured to tissue at the implantation site at operation 1104, and the balloon of implant 110 may be inflated at operation 1106 such that implant 110 is positionally restrained by augment construct 120. In another example of method 1100, implant 210 may be positioned between augment 220 and filaments 226 of augment construct 219 in a deflated condition before augment construct 219 and implant 210 are inserted into the patient at operation 1102. Augment construct 219 may then be secured to tissue at the implantation site at operation 1104, and the balloon of implant 210 may be inflated at operation 1106 such that implant 210 is positionally restrained between augment 220 and filaments 226. In another example of method 1100, implant 210 may be inserted through a cutout of body 410 of receiver assembly 400 and into the internal cavity of body 410 in a deflated condition before receiver assembly 400 and implant 210 are inserted into the patient at operation 1102. Receiver assembly 400 may then be secured to tissue at the implantation site at operation 1104, and the balloon of implant 210 may be inflated at operation 1106 such that implant 210 is positionally restrained within the internal cavity of body 410. In other examples of method 1100, a balloon implant may be inserted into an internal cavity of an augment construct (e.g., cavity 610 of augment construct 599, cavity 631 of augment construct 629, cavity 631A of augment construct 629A, cavity 631A of augment construct 629A, etc.) in a deflated condition before the augment construct and the balloon implant are inserted into the patient at operation 1102. The augment construct may then be secured to tissue at the implantation site at operation 1104, and the balloon of the implant may be inflated at operation such that the implant is positionally restrained between augment and filaments.

Referring now to FIG. 40, an augment construct 629A including an augment 630A is shown according to an example aspect. Augment construct 629A may be substantially similar to augment construct 629, except as shown and described herein, with like reference numbers referring to like elements. As discussed above with respect to augment 630, augment 630A has three closed edges 632A, 633A, 634A and two edges 635A, 636A, surrounding an opening to form an open end 637A of augment 630A and provide access to the cavity 631A. However, unlike the open end 637 of augment 630 shown in FIG. 30B, the edges 635A, 636A are coupled at corners 638A, 639A such that the opening at the open end 637A does not extend all the way from edge 632A to the closed edge 634A. For example, augment construct 629A may include filaments or sutures stitched between edges 635A, 636A to partially close open end 613A of augment 600A. As described below with respect to FIGS. 43-46, the closed corners 638A, 639A may help to retain a balloon inserted into the cavity 631A after the balloon is inflated.

Referring now to FIG. 41, an augment construct 599A including an augment 600A is shown according to an example aspect. Augment construct 599A may be substantially similar to augment construct 599, except as shown and described herein, with like reference numbers referring to like elements. As discussed above with respect to augment 600, augment 600A has two closed edges 602A, 604A and two openings at either end of augment 600A providing access to the cavity 610A. Like the closed corners 638A, 639A of augment 630A, the edges 611A and 612A defining the first open end 613A of augment 600A are coupled together at the corners 614A, 615A, and the edges 616A and 617A defining the second open end 618A of augment 600A are coupled together at the corners 619A, 620A. Similar to the closed corners 638A, 639A of augment 630A, the closed corners 614A, 615A, 619A, 620A may help to retain a balloon inserted into the cavity 610A after the balloon is inflated. Augment construct 599A may include filaments or sutures stitched between edges 611A, 612A and 616A, 617A to partially close open ends 613A and 618A of augment 600A.

Referring now to FIG. 42, an augment construct 599B including an augment 600B is shown according to an example aspect. Augment construct 599B may be substantially similar to augment construct 599A (e.g., with closed corners 614B, 615B, 619B, 620B), except as shown and described herein, with like reference numbers referring to like elements. As discussed above with respect to augment 600A, the edges 602B, 604B of augment 600B are not continuously closed. In the example shown in FIG. 42, the augment 600B includes openings 621B, 622B along the edges 602B, 604B, respectively. Openings 621B, 622B may be smaller than a width of an inflated balloon implant such that the balloon implant, when inflated, cannot slip out of openings 621B, 622B (e.g., without substantially stretching or tearing augment 600B). Augment construct 599B may include filaments or sutures stitched between edges 611B, 612B and 616B, 617B to partially close open ends 613B and 618B of augment 600B.

Referring now to FIGS. 43-46, an example of the method 1000 using augment construct 629A is shown. As shown in FIG. 43, at operations 1002 and 1004, augment construct 629A is delivered to an implantation site and coupled to tissue 654 at the implantation site. FIGS. 44-46 illustrate operation 1006 of method 1000. In FIG. 44, balloon implant 920, including balloon 922 and valve assembly 924, is inserted into cavity 631A through the open end 637A in a collapsed (e.g., folded, rolled, etc.) configuration. For example, valve assembly 924 may be coupled to a shaft of an implant delivery instrument that can be used to guide balloon implant 920 into cavity 631A. As shown in FIG. 45, balloon implant 920 is unrolled or unfolded to an expanded configuration inside cavity 631A. For example, balloon implant 920 may be delivered in the collapsed configuration (as shown in FIG. 44) inside a sheath of an implant delivery instrument, and the sheath may be retracted to expose the balloon implant 920, causing balloon implant 920 to unroll or unfold to the expanded configuration as shown in FIG. 45.

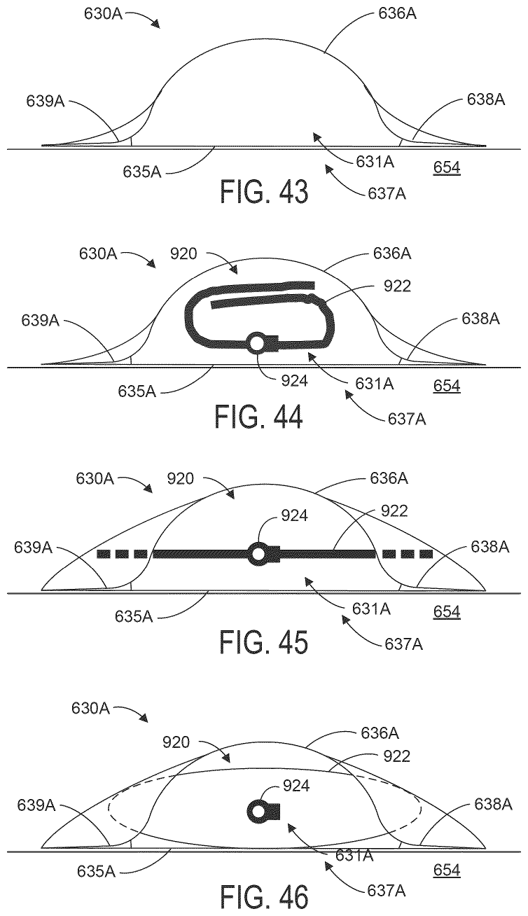

As shown in FIG. 46, balloon 922 is inflated. For example, the shaft of the implant delivery instrument may include a conduit that supplies fluid to balloon 922 via valve assembly 924 before the shaft is decoupled from valve assembly. Because of closed corners 638A, 639A of augment 630A, the opening in the open end 637A of augment 630A may be smaller than the width of balloon 922. Thus, when balloon 922 is inflated, closed corners 638A, 639A serve to retain balloon implant 920 inside cavity 631A, such that balloon implant 920 may not be removable from cavity 631A without deflating balloon 922 or substantially stretching or tearing augment 630A. While FIGS. 43-46 illustrate method 1000 using augment 630A, it should be understood that a similar process may be performed with augment 600A or augment 600B, with the respective closed corners 614A, 615A, 619A, 620A and 614B, 615B, 619B, 620B retaining balloon implant 920 in the respective cavity 610A or 610B.

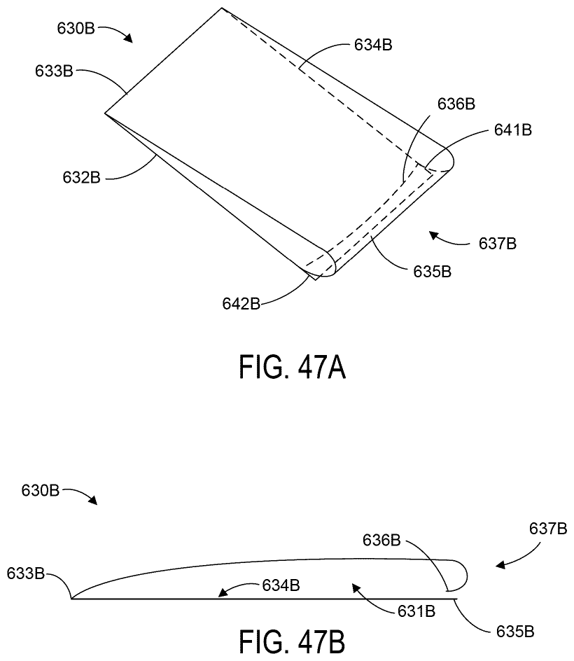

Referring now to FIGS. 47A and 47B, a perspective view and a cross-section of an augment construct 629B including an augment 630B are respectively shown according to an example aspect. Augment construct 629B may be substantially similar to augment construct 629, except as shown and described herein, with like reference numbers referring to like elements. As discussed above with respect to augment 630, augment 630B has three closed edges 632B, 633B, 634B and two edges 635B, 636B, surrounding an opening to form an open end 637B of augment 630B and provide access to the cavity 631B. Edge 636B of augment 630B is curved inward toward cavity 631B, which may allow edge 636B to help retain a balloon inside cavity 631B. Each end of edge 636B, where edge 636B meets edges 632B, 634B, may be coupled to the respective edge 632B, 634B (e.g., forming a flap similar to an envelope closure of a pillowcase) so that edge 636B remains curved inward toward cavity 631B. For example, the edge 636B may be folded over and coupled (e.g., stitched, coupled with adhesive, etc.) to edge 632B, 634B along corners 641B, 642B. The flap formed by edge 636B may be lifted to insert a balloon implant into the cavity 631B and then lowered such that the balloon implant is retained within cavity 631B by the flap.

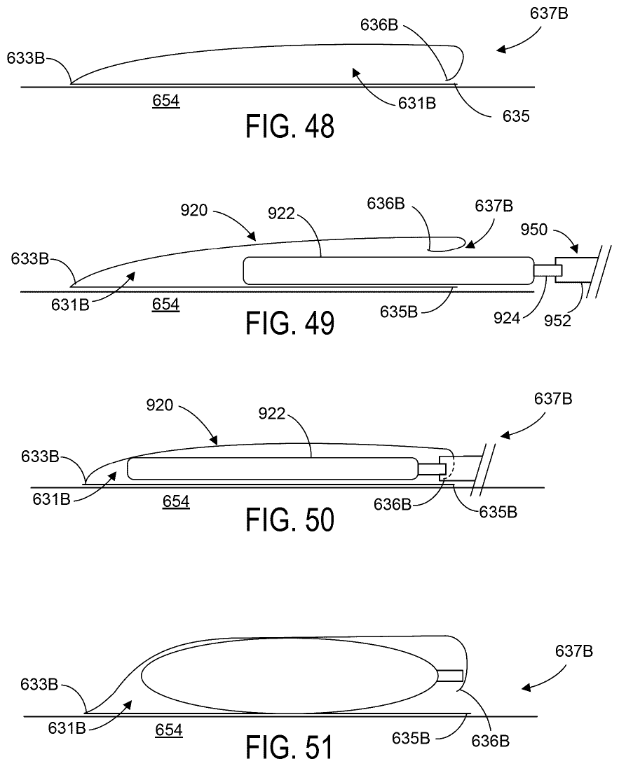

Referring now to FIGS. 48-51, an example of the method 1000 using augment 630B is shown. As shown in FIG. 48, at operations 1002 and 1004, augment 630B is delivered to an implantation site and coupled to tissue 654 at the implantation site. FIGS. 49-51 illustrate operation 1006 of method 1000. As shown in FIG. 49, balloon implant 920, including balloon 922 and valve assembly 924, is inserted into cavity 631B through the open end 637B in a collapsed (e.g., folded, rolled, etc.) configuration. For example, valve assembly 924 may be coupled to a shaft 952 of an implant delivery instrument 950 that can be used to guide balloon implant 920 into cavity 631B. As shown in FIG. 50, balloon implant 920 is unrolled or unfolded to an expanded configuration inside cavity 631B. For example, balloon implant 920 may be delivered in the collapsed configuration (as shown in FIG. 49) inside a sheath of implant delivery instrument 950, and the sheath may be retracted to expose balloon implant 920, causing balloon implant 920 to unroll or unfold to the expanded configuration as shown in FIG. 50. In some examples, edge 636B may be continuous such that edge 636B deflects or stretches around shaft 952. In other examples, edge 636B may include an opening or notch allowing shaft 952 to extend through edge 636B without edge 636B stretching or deflecting around shaft 952.

As shown in FIG. 51, balloon 922 is inflated. For example, the shaft 952 of the implant delivery instrument 950 may include a conduit that supplies fluid to balloon 922 via valve assembly 924 before shaft 952 is decoupled from valve assembly 924. The curved edge 636B of augment 630B retains balloon implant 920 inside cavity 631B. As discussed above, edge 636B may include an opening or notch. When shaft 952 is decoupled from valve assembly 924, valve assembly 924 may extend through the opening or notch such that edge 636B envelops balloon 922, retaining balloon implant 920 in cavity 631B.

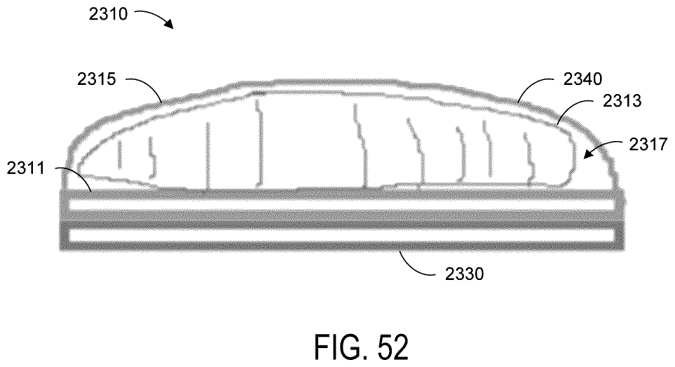

Referring now to FIG. 52, an augment construct 2310 is shown according to an example aspect. As discussed above, in some examples, an augment may be formed into the various structures discussed herein that are configured to receive a balloon implant in an inner cavity (e.g., augment 600, augment 630, body 410 of receiver assembly 400, etc.), while in other examples, the augment may instead be coupled to a separate retention structure defining the inner cavity. In the latter examples, the augment may be made of a first material configured to promote repair of the joint (e.g., collagen), while the retention structure defining the inner cavity may be made from a second material that may not be configured to promote repair of the joint but that may have material properties that improve retention of the balloon implant (e.g., flexibility, durability, etc.), such as a polymer. In various aspects, the retention structure may be mechanically coupled (e.g., using filaments, sutures, staples, etc.) or chemically (e.g., adhesively) coupled to the augment to form the complete augment.

As shown in FIG. 52, augment construct 2310 includes an augment 2330 and a retention structure 2340. Augment construct 2310 may be substantially similar to the other augment constructs discussed herein, however, retention structure 2340 may be manufactured by electrospinning material (e.g., polymer material) onto augment 2330 (which may be made, e.g., of collagen). In the example shown in FIG. 52, a base layer 2311 of retention structure 2340 is applied to augment 2330 using electrospinning. A mold form 2313 may then be placed on the base layer 2311. Additional material may then be applied all around the mold form using electrospinning to form the body 2315 of the retention structure 2340. The mold form 2313, which may be made of a water soluble material (e.g., a salt), may then be removed to form an inner cavity 2317 of retention structure 2340. For example, water may be inserted into an opening in retention structure 2340 to dissolve mold form 2313 to form inner cavity 2317. By using electrospinning to form retention structure 2340 directly on augment 2330, no adhesive may be required to bond retention structure 2340 to augment 2330, and the manufacturing process may be simplified. As discussed above, retention structure 2340 may be formed in the shape of any of the augments disclosed herein (e.g., augment 600, augment 630, body 410 of receiver assembly 400, etc.), such that a balloon implant may be inserted into inner cavity 2317 and retained as discussed above.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A method of delivering a balloon implant into a patient, the method comprising:
delivering an augment construct to an implantation site within the patient, the augment construct comprising an augment;
securing the augment construct to tissue at the implantation site; and
delivering a balloon implant comprising a balloon to the implantation site and inflating the balloon such that the augment construct positionally restrains movement of the balloon implant when the balloon is inflated.

2. The method of claim 1, wherein the augment construct includes a plurality of filaments, each filament including ends attached to the augment, wherein the movement of the balloon implant is restrained by the plurality of filaments or the plurality of filaments in combination with the augment.

3. The method of claim 1, wherein the augment construct defines a cavity and one or more openings large enough to allow passage of the balloon implant in a collapsed configuration into the cavity, wherein delivering the balloon implant comprises inserting the balloon implant into the cavity via a first opening of the one or more openings.

4. The method of claim 3, wherein the balloon is inflated to a size that restricts the removal of the balloon implant from the cavity through any of the one or more openings.

5. The method of claim 3, wherein the one or more openings comprise at least two openings.

6. The method of claim 3, further comprising closing the first opening after inserting the balloon implant into the cavity.

7. The method of claim 1, wherein the augment construct defines a cavity and includes only a single opening suitable for delivering the balloon implant therethrough into the cavity, wherein delivering the balloon implant comprises inserting the balloon implant into the cavity through the opening.

8. The method of claim 7, further comprising closing the opening after inserting the balloon implant into the cavity.

9. The method of claim 8, wherein closing the opening comprises tensioning a filament.

10. The method of claim 9, wherein tensioning a filament comprises stitching the opening shut.

11. The method of claim 1, wherein the augment has a balloon-type structure with a plurality of peripheral cutouts, each cutout spaced apart from the other cutouts, and wherein the balloon- type structure defines a cavity accessible through each of the plurality of cutouts, wherein delivering the balloon implant comprises inserting the balloon into the cavity through one of the plurality of cutouts.

12. A prosthesis comprising:
an inflatable balloon implant; and
an augment construct securable to tissue at an implantation site and comprising an augment, the augment construct defining a receptacle configured to receive the balloon implant at the implantation site in a deflated condition and to restrain movement of the balloon implant after the balloon implant is inflated at the implantation site.

13. The prosthesis of claim 12, wherein the augment construct comprises a plurality of filaments, each filament including ends attached to the augment, the augment and the plurality of filaments defining the receptacle therebetween.

14. The prosthesis of claim 12, wherein the receptacle is a cavity accessible from a plurality of openings in the augment construct.

15. The prosthesis of claim 12, wherein the receptacle is a cavity accessible from exactly two openings sized to receive the balloon implant in the deflated condition on opposite ends of the augment construct, the augment comprising closed edges extending between the two openings.

16. The prosthesis of claim 12, wherein the receptacle is a cavity accessible from exactly one opening sized to receive the balloon implant in the deflated condition.

17. The prosthesis of claim 12, wherein the augment includes a plurality of peripheral cutouts, each cutout spaced apart from the other cutouts, wherein the receptacle is a cavity accessible through each of the plurality of cutouts.

18. A prosthesis comprising:
an inflatable balloon implant;
an augment securable to tissue at an implantation site; and
a structure attached to the augment, wherein the structure or the structure in combination with the augment defines a receptacle configured to receive the balloon implant at the implantation site in a deflated condition, and wherein the structure is configured to restrain movement of the balloon implant after the balloon implant is inflated at the implantation site.

19. The prosthesis of claim 18, wherein the structure comprises a plurality of filaments, each filament including ends attached to the augment, and wherein the balloon implant is restrained by the plurality of filaments or the plurality of filaments in combination with the augment after the balloon implant has been inflated.

20. The prosthesis of claim 18, wherein the structure comprises a receiver assembly with a plurality of peripheral cutouts, each cutout spaced apart from the other cutouts, and wherein the receiver assembly defines a cavity accessible through at least one of the plurality of cutouts, wherein delivering the balloon implant comprises inserting the balloon into the cavity through one of the plurality of cutouts.
